(19) United States
(12) Patent Application Publication
(10) Pub. No.: US 2018/0209978 A1
(43) Pub. Date: Jul. 26, 2018

(54) UNIVERSAL, NON-INVASIVE, EARLY DETECTION SYSTEM FOR CANCERS

(71) Applicant: Richard Postrel, Miami Beach, FL (US)

(72) Inventor: Richard Postrel, Miami Beach, FL (US)

(21) Appl. No.: 15/880,527

(22) Filed: Jan. 25, 2018

Related U.S. Application Data
(60) Provisional application No. 62/450,328, filed on Jan. 25, 2017.

(51) Int. Cl. G01N 33/574; A61K 35/765

(52) U.S. Cl. CPC G01N 33/574; A61K 35/765

(57) ABSTRACT

The present invention provides a universal system that detects cancer cells at all stages including early developmental stages as well as matured cancerous tumors. The system relates to monitoring the environment surrounding abnormally metabolizing cells, e.g., predominantly glycolytic cells, cancerous cells, precancerous cells, and/or cells with reduced mitochondrial Electron Transport Chain activity. Nanosensors detect chemical signatures of the organic compounds released by cancer cells into bodily fluids or to the air to detect cancerous or precancerous metabolisms in samples such as urine, saliva, sweat, blood or breath. Treatments provided in recognition of the signature metabolic changes occurring in cancer cells target the abnormal metabolism generally with special emphasis relating to changed mitochondrial metabolism and cell survivability. The treatments include, but are not limited to: therapeutic compositions and methods for treating, delaying, slowing or preventing one or more cancers.

### BACKGROUND

The present invention provides a universal system that detects cancer cells at all stages including early developmental stages as well as matured cancerous tumors. Nanosensors detect chemical signatures of the organic compounds released by cancer cells into bodily fluids or to the air. The invention addresses the metabolic shift as a cancer matures from an emphasis on the efficient mitochondrial sourced oxidative phosphorylation production of ATP to the less efficient cytoplasmic glycolysis. As metabolism changes to favor glycolysis, volatile organic carbon compounds show a changed pattern. Treatments associated with recognition of the signature metabolic changes address the abnormal cellular metabolism generally with a special emphasis on the relation between cancer and mitochondrial activities that control cell survival. These associated treatments include, but are not limited to: therapeutic compositions and methods for treating, delaying, slowing or preventing one or more cancers.

Cells, including cancer cells, can be eliminated (removed from an organism) by a programmed death process called apoptosis or cells may face necrosis through other paths. Energy conversion pathways are a prime target of therapy. For example, for a cell to grow, one embodiment of chemical energy, including, but not limited to: an amino acid, a triglyceride, a carbohydrate, a sugar, etc., must be converted into another chemical to build cell structure. All chemical reactions for accomplishing these building tasks require energy input—at least to overcome the transition state. Machinery to provide the fuel to energize reactions resides to some extent in the cytoplasm, but predominantly is found in one of the cell's organelles, the mitochondrion.

Mitochondria are the site of oxidative phosphorylation (OXPHOS) within eukaryotic cells. OXPHOS is by comparison to alternative reaction pathways, an extremely efficient process though which eukaryotic cell metabolisms produce ATP, the major energy source for driving chemical reactions within the cell. Since all reactions consume energy in the cell, even cells in a disease state require a functioning energy metabolism. It stands to reason that when a cell's functions and chemical reactions are altered by disease, the energetic background will change or compensate in some manner. The disease process will manifest in a different metabolic reaction profile characteristic of that disease. This is easily understood in the example where a virus inserts its genes into the cell and thereby commandeers the cell's synthetic operations to manufacture new viral particles.

As cancers develop, multiple mutations occur in the nuclear DNA (nDNA) of the cell. Each mutation can change how proteins encoded by the nDNA are expressed and processed into cell protein. These mutations lead to the appearance of developed cancer. As the cancer conditions are developing, other chemical processes within the cell are adapting to support the changes underlying the cancer's pathways. Before cancer is readily observable, these metabolic changes are transpiring in the background.

Mitochondrial DNA (mtDNA) mutations are also observed, some silent, others active. In one aspect, the invention concerns compounds that are specific to mitochondrial genomic material that has been mutated as part of the cancer's developmental path. Anti-sense complements or otherwise mutation specific compounds, by interacting with the cancer related mutated mitochondrial genome, will thwart the metabolism in mutated mitochondria and induce apoptosis or other death mechanism of cancerous cells. The invention also features recruitment of mitochondria and mitochondrial activities to drive death of the cancer cell in which the mitochondria reside.

Most organisms on earth are aerobic; they use oxygen (O₂) as a driver in their metabolisms. Even plants which produce O₂from sunlight use the O₂ for their own metabolic functions. In eukaryotic cells, mitochondria are major producers of a predominant fuel of chemical energy in the form of adenosine triphosphate (ATP) which is used for modifying proteins (e.g., phosphorylation reactions, synthesis reactions, cleavage reactions, etc.), such as enzymes, and also for providing the energy source underlying most enzymatic reactions within the cell. Although fatty acyl CoAs and several alternative chemical energy sources can be used in metabolism, the common simple sugar, glucose, is a preferred source. The oxidative phosphorylation pathway residing in mitochondria is a comparatively very efficient transformer of glucose energy to ATP energy. Using mitochondrial oxidative phosphorylation (OXPHOS), mitochondria can convert the energy of one glucose molecule to 36-38 ATP molecules. By contrast, the glycolytic pathway occurring in the cytoplasm, and which does not require O₂, produces only 2 molecules of ATP (and two molecules of lactate) for each glucose molecule consumed.

These ATP generating biologic reactions tend to be exothermic—as are most chemical conversions. Accordingly, production of ATP generates heat. Understanding that glucose has a Gibbs free energy value of around 2800 kjoule/mol and ATP about 30, indicates that the heat generated by efficient mitochondria using one mol (180 grams) of glucose would be about 1800 kjoules/mol glucose or about 50 kjoules/mol ATP. Using the less efficient glycolysis pathway that is dominant in neoplastic cells releases approximately 20 times the heat into the cell for each ATP molecule produced or about 1000 kjoule/mol ATP.

Cancer cells are in part characterized by their predominant reliance on glycolysis rather than the more efficient mitochondrially driven OXPHOS and thus must generate more heat to continue functioning even in the absence of increased metabolism the cancer cell requires to support its rapid growth and proliferation. Other features being equal, for example, a similar ability of the system to conduct or convex heat from the cells, means that less efficient and higher metabolic rate cells, such as cancer cells, will run hotter than normally metabolizing cells.

Elevated temperature is not the only observable characteristic of cancerous or precan-cerous cells. As discussed below, several physical characteristics, including resonance of atoms of interest, is detectable using diagnostic tools like MRI. MRI is expensive because of the purchase price of the necessary machines and their operations and maintenance costs. But MRI scans can help with early treatment. Gas chromatography and mass spectrometry can also be used to detect signature patterns of volatile outputs from the cells whose metabolisms are changed in a cancerous direction. Unlike MRI, no localization within the body is possible, but though expensive, these machines are not as expensive to operate as MRIs.

Other aspects of differentiated metabolism can be identified with less expensive sensors and alarms. For example, the common biology laboratory fly, the fruit fly (drosophila), uses its antennae as sniffer organs. Specialized cells on the antennae detect “odors”. In a 2013 paper Strauch et al report that fruit fly response vectors are reliable indicators of chemicals, such as volatile organic hydrocarbons (VOCs), found in many consumer products, but also emitted by cancer cells. Such devices can be configured to measure breath contents or evaporated components from liquids such as blood and urine. A man-made version of the fruit fly antennae, electronic gas sensor arrays, are now available for other purposes, e.g., workplace safety, and are now being modified to non-invasively detect nano marker patterns indicative of aberrant cancerous or precancerous metabolic output. Though the fruit fly may be one of the least expensive and most easily analyzed (Every neuron can be monitored.), other animals have been shown to have cancer detection ability. Dogs, mice, honeybees and the Giant African Pouched Rat exemplify animals that have been successfully trained for disease detection.

When the organism is simple, such as the fruit fly, or the slightly more complex honeybee, tests of which neurons fire during detection can help humans identify the nano marker compound patterns or perhaps an individual nano marker compound indicative of cancer development. Electronic arrays based on these insect responses have been well received in industrial applications. The differentiated metabolism of the precancer or cancer cells is responsible for multiple effects that can be observed or measured by these animal or electronic sensors. These effects include emission of metabolic products that produces a chemical signature hereinafter termed “nano marker” for convenient reference. Other consequences of the differentiated metabolism, e.g., resultant increased local temperature or localized expression of a chemical/biochemical marker or increased concentration of a growth and/or proliferation emphasis, are also useful signal features in practice of this invention. By screening for disease and then using, e.g., the heat of increased metabolism to localize, to target and to switch on at the active site, an early round of delivering the therapy compositions can be accomplished in the absence of grossly observable symptoms.

Canines, pigs, giant rats and other larger animals have been reportedly trained to detect one or cancers including lung, breast, prostate and bladder cancers. A 2011 study reported that trained dogs were able to detect lung cancers and distinguish it from other diseases such as COPD with better accuracy than currently available tests. Specific aldehydes, ketones alcohols, amino alcohols and polyamines are candidate compounds that may be detectable by the sensing animals. The chemical components and/or their ratios potentially sensed by the animals is not known. Simpler organisms, such as the fruit fly whose nerves are each accessible to monitoring reportedly respond differently when exposed to different combinations of volatile organic compounds (VOC)s, such as aldehydes, alcohols and ketones. Different cells with their different VOC outputs were distinguishable. In this study different cell types and cancer versus non-cancer cells could be differentiated by analyzing the fruit fly neuronal stimulation patterns. Other simple laboratory organisms such as C. elegans and several moths have had complete mapping of their olfactory nerves and thus are available as tools for detection of VOCs and other compounds (hereinafter simplified to VOCs).

The present invention provides a system for screening cancer in general and detecting one or more cancer types within an individual or even a defined population.

The process features monitoring, for example fruit fly antennae that have been determined to be responsive. A first embodiment of the invention features monitoring the patterns of nervous responses of small nerve arrays (bioarrays), such as insect, roundworm, etc., to determine specific VOCs or patterns of VOCs correlating with a cancer. Repeating monitoring using other cancers can be used to expand the library of signatures characterizing specific cancers. A bioarray may comprise an isolated or intact nerve network comprising one or more sensory nerves—nerves that change firing rate in response to external stimulation—and nerves that change firing rate in response to changed firing rate of another network nerve. A bioarray from similar simple organisms such as the fruit fly can be considered identical to or to be the same as each similarly obtained bioarray. Thus repeated contacting of a bioarray may comprise contacting the same physical array or may comprise contacting a plurality of bioarrays that are considered to be identical to one another.

One or more of these libraries are then used for designing nanosensor chemical arrays whose outputs are dependent on presence of the relevant VOCs and thus are able to detect the presence or absence of one or more cancer signatures. Such sensor array may be specifically designed for sensitivity to one cancer or may include components capable of detecting and identifying multiple signatures. The arrays necessarily do not require limitation to cancer detections, but could be applied to other disease such as diabetes, Crohn's, hepatitis, pancreatitis, lung diseases, renal diseases, etc. Since all diseases involve altered metabolisms of at least one group of cells, then detection aspects of the present invention would be applicable thereto.

VOC contents are adjusted to elicit responses mimicking those obtained when a bioarray is in proximity to a cancerous or precancerous cell. Using one or more of these adjusted VOC exposures, sensor arrays are refined to increase specificity and simplify detection. Nanosensor array outputs are delivered to a processor that compares the outputs to the signature libraries, possibly adjusts one or more sensors of the array to refine sensitivity, analyzes the outputs, including response ratios from different sensors and indicates the disease(s) or a weighted probability of disease(s) of interest.

Alternatively, electronic nanosensors specific at first for classes of VOCs are exposed to a variety of cells including from various organisms; from various tissues; primary cells from different aged persons; primary cells from persons of varied diets, occupations and cultures; cells from varied diseases including several cancers; etc. (This preliminary screening is not necessary but can serve to reduce the number of different sensors to be manufactured and screened. A random approach is also a possible embodiment where the nanosensors ore manufactured with random sensitivities. Those responsive sensors are then analyzed and duplicated for further testing and refinement. As artificial intelligence improves, this random method is expected to become more economical.) As chemical class is associated with specific diseases or cancers, the nanosensors are refined to detect greater specificities within the chemical classes. Cancers will be distinguishable from other diseases based on not only the presence of specific compounds and/or their close derivatives or metabolites, but more precisely by their ratios of the several relevant VOCs.

Signature patterns for several cancers are thereby developed. And since cancers, though differentiated by their characteristic mutations and thus specific to tissues and favored treatments, all share basic cancer characteristics, a next stage for screening is embodied in another aspect of the present invention. As the various cancer signatures are compiled, a pattern recognition software statistically refines the outputs and tests and retests and eventually refines a signature or set of signatures that are applicable as a screening tool for cancers generally.

The nanosensors can be used in several environments. For example, sensor arrays may be configured to monitor primary cells such as from a biopsy, cells cultured from a biopsy, blood samples, urine samples, saliva samples, breathe samples, etc. In a general configuration, a sensor array may be configured to monitor samples obtained from a population, such as a population in a room or passing through a hallway. Information of this type might be used for assessing exposure or results of exposure to radiation or chemical carcinogen, to observe cancer incidence differences in different localities, industrial plants, office environments, etc. and thus be a valuable public health tool useful for prevention or targeting of prevention or treatment efforts.

This differentiated metabolism (higher in neoplastic cells) has been used to identify areas within the body by monitoring glucose uptake, for example using positron emission tomography (PET) scanning. More refined sensors, extremely small biosafe electronic needles and probes that are under development and testing, may improve ability to finely monitor O₂ use and local temperature effects. Heat may not only serve as an indicator of neoplastic like cellular activities, but when supplemented, amplified, complemented or boosted can be used as a target whereby a composition or device can elicit anti-neoplastic actions at or within the targeted cancer cells.

Conversion of chemical energy to other chemical forms or to heat is an essential process of living. This is why we, like all living organisms, consume foods so that these foods can be used to support the metabolism necessary for all our activities. Metabolism converts the food to the chemicals we use to grow and survive. The universal byproduct of most chemical conversions, heat energy, can be observed as an increase in temperature around the site of chemical activity.

Increased ambient temperature is recognized as a diagnostic method indicative of metabolic activity. For example, infra-red sensors that detect heat are used to show locations of living organisms. As organisms, we have evolved to take advantage of the heat produced by metabolism to maintain optimal temperatures for the enzymes that control our metabolisms to sustain life. Different enzymes have different temperatures they need for maximal rates of reactions. As homeothermic organisms evolved, their body temperatures and enzymes evolved together to produce the combination of attributes that each organism and each part of the organism expresses today.

We humans understand, as supported by millennia of experience, that altering temperature can be an important method for controlling growth of biologic organisms. For example, cold temperatures tend to slow growth of many molds and bacteria (ice boxes and refrigeration) and higher temperatures (cooking) can inactivate or kill living things. Our bodies have evolved systems where we increase metabolic rates in certain cell in order to maintain our normal body temperature in the winter or even to produce fever in order to detrimentally impact the ability of pathogens proliferate inside us. Every operation within our body has some degree of temperature dependence.

Bachynsky and Roy in US Patent Application 20150056160 acknowledge this including the observations: “Local heat, systemic hyperthermia and fever therapy have been empirically used as effective treatments for malignant, infectious and other diseases since antiquity. Therapeutic hyperthermia was first documented in the Edwin Smith surgical papyrus in the 17th century. B.C. Coley's toxin extracts of Streptococcus erysipelatis (group A streptococcus) and Bacillus prodigiosus (Serratia marcescens) were used to induce fever for the treatment of patients with advanced cancer. The Nobel Prize was awarded for using fever therapy in the treatment of neurosyphilis with the injection of malarial blood. As late as 1955, the Mayo Clinic advocated using malariotherapy or heat therapy for cases of tertiary syphilis “resistant to penicillin”. Long term remissions in patients with inoperable carcinomas that were treated with hot baths and local heat applications have also been reported. Published observations on the disappearance of malignancies such as a soft tissue sarcoma in a patient experiencing high fever due to erysipelas and tumor lysis of Burkitt's lymphomas following malignant hyperthermia during surgical anesthesia are known.

A comprehensive historical review on anecdotal observations and intuitive rational for the empirical use of therapeutic hyperthermia has been published by Myer, J. L.” and “Except for a few exceedingly rare forms of cancer like childhood leukemias and testicular cancer or immune responsive infections, chemotherapy, radiation or drug therapy often do very little except briefly extend survival. One of the major obstacles to “cure” disseminated cancer and infections has been the innate or acquired resistance of tumor cells and emerging microbes to antibiotics, drugs and treatments given in tolerable doses. Escalation of treatments or use of multiple drugs to overcome resistance is invariably prevented by concomitant toxicities or development of multi-drug resistance. Further, in contrast to drugs, which represent a single molecular species that biochemically interact with specific enzymes or receptors of viruses, prokaryotes and eukaryotes, the action of hyperthermia is biophysical and global. Hyperthermia has no specific heat receptors.”

They further discuss effective treatments using heat against bacteria and the potentiating effect of heat used in conjunction with antibiotics. Heat is also effective against some fungi and parasites. Their teachings of clinical benefits of heat continue with anti-viral and anti-cancer successes, e.g.:
“Use of whole body hyperthermia has been reported to cause regression of Kaposi's' sarcoma, clear oral candidiasis, eliminate hepatitis C, cause remission of Varicella-zoster, increase weight gain and improve CD4 lymphocytes counts in patients with acquired immunodeficiency syndrome (AIDS). Dramatic improvement with hyperthermia therapy has been documented in a patient infected with a debilitating Verruca vulgaris and HIV. The FDA has approved clinical trials involving hyperthermia for the treatment of AIDS with a patented extracorporeal blood heating machine to induce whole body hyperthermia. The FDA has recently expanded the extracorporeal heating machine trials to permit treatment of 40 HIV infected patients.”
   and:
“Hyperthermia can augment cytotoxicity and reverse drug resistance to many chemotherapeutic agents. Moreover, hyperthermia has also been shown to enhance the delivery of many novel cancer therapeutic agents, i.e., monoclonal antibodies to neoplasms with resultant improvement in antitumor effect; enhance the delivery of gene therapy with use of viral vectors; and, augment drug delivery and antitumor effects when using drug containing liposomes. In addition to increasing the rate of extravasation of liposomes from the vascular compartment by a factor of 40-50, hyperthermia can also be used to selectively release chemotherapeutic agents from liposomes designed to be thermosensitive. Thermosensitive liposomes are small vesicles composed of lipid phosphatidylcholine moieties constructed to contain and transport a variety of drugs. The liposomes are designed to remain stable in the blood and tissues at physiologic temperatures. When passing through an area of heated tissue however, they dissolve and effectively release their encapsulated contents. Thermosensitive liposomes are used to entrap and carry drugs whose systemic toxicity is desired to be limited to a particular heated tumor, organ or tissue. Examples of drugs that have been encapsulated into liposomes include methotrexate, doxorubicin, amphotericin B, cisplatin and others. Liposomes can be designed so as to release their contents at pre-determined temperatures.”

Recognizing this history of elevated temperature having seemingly miraculous curative powers, external devices to heat the body or a portion thereof have been proposed as therapeutic medical devices. However, external devices have not been popularly accepted because whole body heating, such as in a hot bath, heats the body unevenly and unpredictably. For example, attempting to treat a hematopoietic disease such as a blood cancer might require toxic levels of heat exposure to other body parts, such as liver, in raising temperature of bone marrow to effective therapeutic levels. Another consideration is that localized heating would miss metastatic cells that may have deposited in other regions of the body. Micro- and now nano-sensors have seen proof of principle and are being engineered specifically for several biologic applications including localized heating.

Using external devices for targeted delivery of heat has been tried, but invasive treatments tend to have risks and high costs. One approach has been to use nanoparticles as a sort of antenna to receive electromagnetic radiation that will heat tissue where the particles are deposited. Methods for selective delivery of these particles to cancers or virally infected cells are critical for success of these techniques. For cancer and other applications the risks of treatment may be overwhelmed by risks of continued disease development.

Completely non-invasive ultrasonic excitation while when tuned properly is effective at selectively delivering heat energy, but it suffers from poor focusing power and heats healthy as well as targeted cells. Microwaves as a heat generating source are ineffective generally more than two inches into biologic tissue.

### BRIEF DESCRIPTION

Evolution has progressed from the early stages of life, from simple replicating molecular entities to larger frameworks now embodying cells which cooperatively act to form complex multicellular organisms. Within complex organisms, for example, a human, cells perform different specialized functions; serotonin receptors are not predominant in liver cells; hemoglobin is not the predominant protein in a retinal cell. But each cell at its nascence had a progenitor cell that could follow any path. As the single progenitor matured, it divided to branch off many different branches that further branched to form every cell of every type and function necessary to maintain the multicellular organism.

In mature cells a large proportion of functions available in the progenitor and passed down in the genomic material that every cell carries are turned off. Sometimes however, this switching off mechanism malfunctions. And during normal development as physical building tools are no longer needed they needed or are only transiently needed (think: baby teeth or a mother's nursing equipment) it is advantageous to the organism to dispose of cells and their functions that are no longer useful.

Evolution has selected survival benefits relating to the ability of individual cells that are part of the organism to remove their burden on the whole organism's physical and metabolic limitations. Cells of complex organisms possess pathways that when activated set in motion a set of reactions within the cell that decompose the cell from the inside resulting in a safe (for the organism) non-inflammatory cell death. In contrast, when a cell is removed by necrosis, outside events such as physical trauma, toxic chemical, burning, immune system attack, temperature, membrane destruction, etc., lead to a less elegant cell death that may or may not benefit the organism.

When a cell recognizes that critical mechanisms including its switching off mechanism have malfunctioned or that its functions are no longer being turned on, for example, activation of a gene expression initiated by occupation of a hormone receptor on the cell's plasma membrane, the cell has several mechanisms that can be initiated to bring about an orderly deconstruction. As cells age many will become damaged in ways that are not repaired. In these cases it is a normal life function for these cells to self-initiate apoptosis so that they can be replaced by a more useful healthy progeny from one of the retained stem cells.

When the switching off mechanism malfunctions, metabolism is detrimentally affected and the malfunctioned cell expresses an abnormal metabolism, generally this triggers apoptosis and expediently removes the malfunctioning cell. But in rare, but not rare enough cases, the aberrant metabolism and the hosting cell remain in the organism, consuming its resources without appropriate contribution to the whole organism's well being. In severe cases, these aberrant cells continue to grow and proliferate, forming physical masses that impact the health of surrounding cells and the organism as a whole. In these cases where the cells are a) not recognizing their maladaptations, b) failing to undergo apoptosis and c) further continuing to act in a manner of stem cells and to proliferate in a misguided attempt to restore functions of the malfunctioning cell to the whole organism, a massive tumor can result. Possibly because the organism's system recognizes a missing output and sends hormones or cytokines to encourage the damaged cells to fill the gaps, these cells may proliferate at an accelerated pace. Correcting these rare instances so as to undo tumorogenic activity and allow healthy cells to continue to support the organism is one ultimate goal of the present invention.

If the cells were behaving like the great majority of cells do and were performing metabolic functions in support of the whole organism, there would be no concern. But when these cells function abnormally, the abnormal functions are rooted in enzymatic (or chemical) reactions that are not in the organism's best interest. These reactions may eventually produce obvious manifestations, but the maladaptations on the individual molecule or nano scale must come first. These abnormal reactions will have several effects. First, they may produce compounds that are not normally made by the cells, for example when an incorrect enzyme is expressed. Second, they may produce excess amounts of a metabolite for example when an alternative pathway is used or a subsequent reaction is not taking place. Third, they may be consuming resources at a rate faster than healthy for the organism and thereby starving healthy proximal cells, or the metabolites released to neighboring cells may cause these cells to alter their metabolisms and outputs in response. Fourth, the cells may not metabolize wastes from their own cell lineage or from the organism in general thereby using other means of disposal, such as sweat, urine, breath; or another detoxification pathway within the organism with its abnormal metabolite(s).

These adaptive events, especially at early stages, would not be apparent to a casual outside observer. But the nano scale events are sensed by the enzymes, ligands and/or receptors in that or a nearby cell. If the organism was not affected by these events, its health would not be affected on the larger scale and no therapy would be required. The trick therefore is to scale down the therapeutic process to these extremely localized molecular events, i.e., to rely upon a signal from nano scale sensors, or nano sensors for short.

These nano sensors will sense presence of signs that are not casually observable. For example, minor temperature variations, and possibly including their minor metabolic effects, nano signatures in, for example, blood, urine, sweat or breath. As an analogous kind of thought, diabetes can be detected by a sweet tasting urine, an increased water intake, increased urination, a breath with fruity or ketone odor, a measurement of the amount of glucose in the blood, an assay of circulating insulin, an assessment of function or insulin receptor, blindness, poor circulation, etc. On a grand scale before, for example, ketosis could be recognized as a sign of diabetes, death from diabetes or circulation problems had to be recognized. Now we can treat diabetics before serious damage by sensing one of these associated signals. The present invention accomplishes similar outcome by sensing on a nano scale events that will detrimentally affect the organism if they are left to continue.

The present invention—though many of its parts can be considered separate or sub-inventions—in its grandest form applies nano sensing technology, either non-invasively, for example, by sensing breath, urine, retinal scan, etc., or by using nano probes given a physical presence within an organism or in specific adaptations in a selected location within the organism. The selected location may be in the vicinity of the suspected tumor or might be at another site, perhaps where a metabolite of the abnormal cell would be further metabolized: for example, liver, kidney, or simply in a blood vessel. Sensing of metabolic outputs such as chemical products and heat are two important applications of this nano sensing technology and its application to arresting abnormal metabolism and the cells responsible therefor.

Organisms have evolved with differing core temperatures and different temperature gradients between core and extremities. This evolutionary process includes natural selection of species specific enzymes, each with optimized activities at the temperatures, ion concentrations, pH, etc. that evolution has determined. The effects of heat/temperature are widespread, affecting many enzymes' and cells' activities to different extents. No one molecular event appears responsible for the beneficial outcomes observed when pathogens are exposed to elevated temperatures. Evidence of widespread heat effect is seen in the heat shock proteins produced by bacteria under heat stress. These heat shock proteins have evolved to rescue the microorganism and cells in macroorganisms from denaturing effect of heat on many cell enzymes, perhaps by preventing heat from unfolding the proteins to which the shock proteins are bound, by restoring proteins to their active 3-dimensional configuration or by assisting destruction of non-restorable damaged proteins. It is thus apparent that heating inside a cell is capable of stressing a cell—possibly even to the point of that cell's demise.

Heating, by causing non-specific damage, is observed as initiating or facilitating apoptosis, an orderly cell death, in heat stressed cells. Accordingly, heating in general areas may be advantageous in its ability to support apoptosis in stressed cells when killing these cells is desired. But advantageously, heat generated within a cell or a cell organelle, being specifically localized would provide an optimized localized promotion of apoptosis in that specific cell.

Apoptosis is an important process in an organism' development. If a cell simply dies, for example by osmotic rupture of the membrane, the contents are spewed about, in effect littering the area and causing physical and chemical challenges to surrounding tissues. Apoptosis is an orderly process of cell degradation that has evolved to prevent these outcomes. As part of living, unneeded or unhealthy cells undergo a controlled removal process termed “apoptosis”.

During apoptosis “caspase” enzymes are activated, DNA and proteins are digested and phagocytic cells are recruited to harvest the apoptosed cell. Cases are cysteine proteases that cleave proteins after aspartic acid (abbreviated Asp or D) residues. Caspase activation allows these enzymes to tear down the cell's internal structures, including the cytoskeleton and other proteins. Phosphatidylserine is moved to the cell's outer membrane and becomes accessible for recognition by macrophages and other recruited phagocytic cells. DNAses become activated to break down DNA into sub kilobase fragments. The result is cellular destruction by either apoptosis or necrosis.

Often cell stress, such as DNA damage from any source, oxidative stress, hypoxic stress, cytoskeleton malfunction, heat stressed or otherwise unfolded proteins, removal or presence of hormonal signals, a leaky membrane (especially to Ca⁺⁺), etc., will initiate an apoptotic event. When mitochondrial membrane integrity is compromised, e.g., permeability is increased, cytochrome c and other pro-apoptotic compounds escape into the cytoplasm. Increased permeability if generalized throughout the membrane surface (not through a specific carrier or pore) would also allow passage of ions from the membrane side of higher concentration to the side with a lower concentration of that ion. The ion flow can be characterized as an electrical conductance as electrical charges are moving. The membrane potential, a force that may control concentrations across the membrane of other charged particles would also be degraded.

Although apoptosis is natural and necessary, apoptosis must be limited in order for the organism to survive. Cells contain two groups of proteins that balance between pro-apoptotic activities and anti-apoptotic activities. Proteins that act to prevent apoptosis, for example, by inhibiting cytochrome c that has escaped into the cytoplasm from carrying out its apoptosis initiating effect include but are not limited to: Bcl proteins: Bcl2, BclXI, BclW, and Bfl1 and Mcl1.

Pro-apoptotic proteins in opposition to these Bcl affiliated and other anti-apoptotic proteins include for example: Bax, Bak, Bad, Bcl-Xs, Bid, Bik, Bim and Hrk. These have the general effect of promoting release and activity of cytochrome c to the cytoplasm. Activation states of these proteins are controlled within the cell. The balance between the effectiveness of these pro-apoptotic and anti-apoptotic proteins determines whether the cell initiates and completes apoptosis. The degree to which a cancer line has the balance tilted towards the anti-apoptotic direction, e.g., increased Bcl protein expression and activity or decreased Bak or other anti-apoptotic proteins, varies with cancer types. The balance in any particular cancer at the time of testing can be a consideration for optimizing an individual's therapeutic protocol. One advantage in interfering with Bcl activity is that the affected cells are likely to apoptose thus killing the recipient cell and sparing any non-specific targeted mutations from causing downstream unintended genetic problems such as those seen in early trials of genetic engineering.

One nuclear factor that has been associated with cancer development and metastasis is NF-KB as summarized by Xia et al (Cancer Immunol Res. 2014 September; 2(9): 823-830): “A significant number of human cancers have constitutive NF-κB activity due to the inflammatory microenvironment and various oncogenic mutations. NF-κB activity not only promotes tumor cells proliferation, suppresses apoptosis, and attracts angiogenesis, but it also induces epithelialmesenchymal transition, which facilitates distant metastasis.”

Angiogenesis is necessary to make the blood vessels that can carry nutrition to and metabolites from growing tissue. Rapidly growing tumors where angiogenesis has not kept pace with growth will experience hypoxia. Hypoxia is not just a shortage of oxygen, but is often associated with a build up of metabolites the cell needs to get rid of. These metabolites and the shortage of O₂ which limits generation of ATP using OXPHOS interferes with phosphorylation events necessary for normal enzymatic actions within the cell. The aberrant enzymatic activity also causes permanent damage to proteins, lipids and mitochondrial nucleic acid and or nuclear nucleic acid. Further damage is brought on by the reactive oxygen species (ROS) generated at the time of oxygen stress. The oxidative stress and effect on ATP availability impacts the cell's ability to control Ca⁺⁺ distribution and membrane integrity and permeability.

Leakage of cytochrome c from inside mitochondria has been cited as leading to an apoptotic cascade. Cytochrome c acts in conjunction with SMAC (also released from the outer mitochondrial membrane) XIAP, APAF1, caspase 9, BID, BAX, BAK and other proteins to split and activate caspaces, for example caspase 3 and caspace 8, which then in turn cleaves an inhibitor (DFF45/ICAD) of caspace activated deoxribonuclease (CAD), an endonuclease, which is then set loose to cut DNA.

Although one requirement for a cell to be cancerous is an accumulation of mutation events, the caspase proteins appear intact in most cancers, though reduced expression levels of caspases have been observed. Since caspases are available in most cancers, activation of these to encourage or foment apoptosis is a pathway available for managing most cancer forms.

The mitochondrion is an organelle that often participates in early apoptotic events. Mitochondria are the only mammalian organelle with an integral genome. The human mitochondrial DNA (mtDNA) encodes 14 proteins (Electron Transport Chain (ETC) genes: ND1, ND2, ND3, ND4, ND4L, NDS, ND6, CYB, C01, CO2, CO3, ATP6 and ATPS; and RNR2) and genes for tRNA and ribosomal RNA. Mitochondrial metabolism both supports and is supported by products of the nuclear genome. But the nuclear genome DNA is insufficient for producing all the cells requirements. US application 20160354332 filed Jun. 8, 2016 teaches that inherent to the electron transport chain's key involvement in producing the cell's ATP by making a H⁺ ion gradient across the mitochondrial inner membrane, aspartate is an essential byproduct. Thus a nano signal rooted in mitochondrial dysfunction can be caused by a cellular event and damage to mitochondrial function may contribute to aberrant chemical signatures not apparently directly related to energy metabolism.

The proton gradient across the mitochondrial inner membrane is essential for mitochondrial health and its depolarization can participate in demise of the cells though apoptotic pathways. Thus intact membrane permeability is important for cell survival and depolarization of the mitochondrial membranes is one path leading to cell death. U.S. Pat. No. 7,348,135 to Kristain and Fiskum teaches methods for assaying integrity of mitochondrial membranes which involve isolated mitochondria and a mixture of enzymes comprising alcohol dehydrogenase (ADH), NADH oxidase (Ox), and horseradish peroxidase (HRP), which mixture catalyzes at least two coupled, cyclic enzymatic reactions that utilize an intra-mitochondrial substance to signal membrane porosity and as a corollary teaches means to control mitochondrial leaking.

Mitochondria have particular involvement early in the apoptosis cascade. They appear to be vital coordinators of several of the possible initiation stages of apoptosis. While many extracellular factors have been found to trigger the apoptotic cascade, the mitochondrion is one of the internal components of the cell that when activated or damaged has several paths through which it can initiate apoptosis. Compromised mitochondrial membrane permeability and damage to the mitochondrial DNA are two recognized initiators of apoptotic cell death.

Permeabilization through the outer mitochondrial membrane protein pores is one means by which cells initiate apoptosis. Apoptotic proteins such as cytochrome c can be released through an opened permeability transition pore in mitochondrial membranes. Proteins of the outer and inner membranes coordinate to open a transmembrane potential destroying conductance channel. Osmotic events swell the mitochondrion leading to outer membrane rupture and dissemination of mitochondrial contents characteristic in several apoptotic pathways.

Normally mitochondrial oxidation of glucose is the preferred ATP synthesis path used by cells. But when O₂ becomes less available, ATP production switches to glycolysis. However, cancer cells favor glycolysis and down regulate mitochondrial oxidation pathways independent of the surrounding O₂ concentration. Similar decoupling is observed in pluripotent stem cells which possibly indicates a proliferative advantage associated with deemphasizing mitochondrial ATP generation. Nevertheless, down regulated ETC, when extreme, can have a starvation effect on any cell. Another advantageous use of decoupling ATP production from the mitochondria's electron transport chains is for the production of heat. Homeothermic animals frequently employ this process, which is especially evident in brown fat cells that transform chemical energy almost directly to heat energy. Heat generation increases local temperature and as mentioned earlier may be of use in inducing cancer cell death.

Mitochondrial dysfunction is now understood to underpin many disease states. Defects in mitochondrial DNA are an obvious defect of interest that may underlie mitochondrial dysfunction. But the mitochondrial genome is quite sparse, mainly concerned with OXPHOS. Mitochondrial structure, transport proteins, transcription control, mitochondrial membrane proteins, synthetic proteins, and most mitochondrial enzymes are products of the nDNA. Dysfunction therefore more often than not has an extra-mitochondrial cause. Therefore manipulation of the nDNA expression as well as mtDNA encoded events are both available strategies for manipulating ATP metabolism in the control of growth and proliferation of all cells including precancer and cancer cells.

It is now recognized as related by Mochly-Rosen et al, U.S. Pat. No. 9,243,232 that:
“Mitochondria are organized in a highly dynamic tubular network that is continuously reshaped by opposing processes of fusion and fission (Chan, 2006, Ann Rev Cell Dev Biol, 22:79-99; Liu et al EMBO J. 2009 28: 3074-3089). This dynamic process controls not only mitochondrial morphology, but also the subcellular location and function of mitochondria. A defect in either fusion or fission limits mitochondrial motility, decreases energy production and increases oxidative stress, thereby promoting cell dysfunction and death.”

In 2013 Susanne M Rafelski (BMC Biology 2013 11:71) summarized important characteristics of mitochondrial connections or networks:
“In the absence of any gross defects in internal organization, mitochondria maintain their underlying tubular shape and diameter. These tubules are further organized into a network structure spanning the cell at the micron scale. This network also has a shape, with the connectivity of the network and its distribution within the cell varying within different regions of the cell and from cell to cell. The mitochondrial network can exist at the extremes as a collection of small separate sub-networks or one single, interconnected organelle (see dynamics section below). Mitochondrial networks can also exhibit more subtle variations in their topology -for example, containing different numbers of tubule branchpoints (FIG. 2a). This ‘topological’ aspect of mitochondrial shape can be quantified by considering mitochondrial networks as mathematical graphs with edges (tubules) and nodes (branchpoints connecting tubules). The pure topology of the network can then be measured by considering how many edges are connected at each node. By further assigning a physical location within the cell to each node and along the length of each edge, the network can also be considered a ‘geometric graph’, permitting additional analyses that incorporate the spatial component of the networks to describe the shape of mitochondria at the scale of the entire cell. For example, a more over-connected network would exhibit a greater ‘average degree’ (the number of edges that enter each node, averaged over the entire network) while a network with more long tubules between branchpoints might exhibit a greater average length per edge (average of the lengths of all the edges in the network). Applications of standard network analysis methods will be a useful tool to quantify the micron-scale network morphology of mitochondria.”

Mitogenesis, mitophagy, fusion and fission are important factors for maintaining healthy mitochondrial network and for maintain appropriately metabolizing cells.

Accordingly, one target of cancer treatment could theoretically involve hindering the ability of cancer cell mitochondria to participate in either of these fusion or fission processes and thereby impact general mitochondrial functioning. However, accelerating the fission process in comparison to fusion may be one means through which neoplastic cells can diminish their death through apoptosis. Maintaining joined mitochondria as favored by fusion processes appears to make an apoptotic event more possible. Several proposed rounds for use in practicing the present invention emphasize maintenance of fused mitochondria.

Mitochondria in cells are consistently changing. They are transported by the cytoskeleton to areas of need. They may change from more rodlike to more spherical shapes depending on location within a cell. During these processes, mitochondria may fuse together and may split apart under control of proteins within the cell. Two mitochondrial membrane proteins essential for mitochondrial fusion are mitofusin 1 (Mfn1) and mitofusin 2 (Mfn2) which connect two mitochondrial membranes as the fusion process begins.

On the other side, another essential protein for maintaining healthy mitochondria is Drp1, a primarily cytosolic protein. When bound to a mitochondrion, Drp1 forms a constrictive ring around a mitochondrion to split it into two parts. Drp1 is one of the GTPase proteins in mammalian cells. Drp1 interacts with several proteins including, but not limited to: Fis1, Mff, MiD49 and MiD51, that act on the mitochondrial surface to initiate and control mitochondrial fission. Fission is important for maintaining a healthy mitochondrial population and appears to be necessary for cells to proliferate. Drp1 activated mitochondrial fission is associated with inhibiting apoptosis, a property opposite that of eliminating the individual cell. Thus interfering with activity of any of these proteins may slow fission and maintain mitochondria in a fused state.

Cancer cells are characterized by relatively fewer fused mitochondria with respect to more independent or smaller separate mitochondria than seen in non-malignant cells. Consistent with this observation is a finding that Drp1 expression is elevated in cancer cells and that the fraction of Drp1 phosphorylated at the serine residue at position 616 in Drp1, activated Drp1, is elevated. Apparently, cancer cells increase phosphorylation at this spot with the effect of favoring fission activities.

It is possible to chemically inhibit fission by interfering with Drp1. Mitochondrial division inhibitor 1 (Mdivi 1) is a quinazolinone derivative that selectively inhibits mitochondrial division by blocking dynamin GTPase activity in mammalian cells (IC₅₀=˜50 μM). It has been shown to prevent apoptosis by inhibiting mitochondrial outer membrane permeabilization in vivo and to block Bid-activated Bax/Bak-dependent cytochrome c release from mitochondria in vitro. Cayman Chemical reports that Mdivi 1 has been used to maintain mitochondrial integrity and to prevent cell death in models of pathological conditions including cancer, heart failure, and ischemia and reperfusion injuries.

Another inhibitor of Drp1 is a compound known as P110. The polypeptide P110, DLLPRGT, appears more selective for blocking Drp1/Fis1 interaction than Drp1 interaction with other ligands. [A novel Drp1 inhibitor diminishes aberrant mitochondrial fission and neurotoxicity. Xin Qi, Nir Qvit, Yu-Chin Su, Daria Mochly-Rosen. J Cell Sci 2013 126: 789-802; doi: 10.1242/jcs.114439.]

Delivering one or more Drp1 inhibitors in a cocktail to the cancer cell targets can potentiate other pro-apoptotic interventions.

A cocktail of the present invention may optionally include: an anti-nucleic acid moiety specifically targeted at one or more mutated portions of the mtDNA, a pro-metabolism moiety to increase exothermic chemical reactions within the cell, a nutrient to support increased metabolism, a decoupling moiety to decouple ATP production from the generation of the proton gradient by the ETC, a gene engineering moiety to further alter by directed mutation genes of the targeted mitochondrion, a moiety active in the cytoplasmic region to inhibit mitochondrial fission, a moiety that interferes with the release of growth factors or other signals from the targeted cell, a moiety that interferes with the targeted cells response to extracellular cues, a viral envelope to precisely target delivery of the cocktail, viral envelope to act as a carrier for other moieties, a viral envelope that induces cell surface expression of an immunogenic protein, a moiety that binds Bcl promoter site to interfere with anti-apoptotic protections, a moiety that directs mutation of one or more anti-apoptotic proteins thereby potentiating pro-apoptotic stimuli, a collection of multiple moieties in a single cocktail, a series of cocktails with different modes of activities that would address the situation of neoplastic evolution whereby surviving tumor cells to continue proliferation using newly developed advantageous mutations, etc. The cocktail may be delivered as a single bolus or may be delivered over a period of time using one or more methods of delivery.

The method of delivering one or more cocktails of the present invention is not a constraint. The medical art is replete with teachings for administering substances of interest to a target organism or part of an organism.

Liposomal delivery is one means of moving bulk materials through blood or lymph systems. Liposomes can be simple lipid envelopes for delivering aqueous pages to an intended target. They may be multi-layered with alternating lipid and aqueous layers. Outer layers may differ in chemical makeup from inner layers. An outer layer, for example may be heat or pH sensitive preferably releasing contents when desired temperatures or proton concentrations are contacted. In addition to heat as an activating (liposome disrupting) signal, pH or other chemical signals, including attachment to one or more bio-molecule have been successful. The skilled artisan may choose liposomal delivery as one option in practicing this invention.

Liposomal stability or the opposite, liposome lysis, can be modulated by choosing the components of the bilayer. For example, T_{g} temperature increases with length of alkyl chains in a phospholipid and decreases with the presence of unsaturated bonds. Additional lipids, such as cholesterol can be used to modulate T_{g} and to control permeability. Charge effects at various pHs can be selected for example, by choosing an appropriate R group, for example, choline, serine, ethanolamine, glycerol, etc.

Where appropriate, such as for conditions found at the strongly acidic mitochondrial membrane regions, chemicals or small particles sensitive to [H⁺] are useful. pH sensitive paramagnetic particles, nanoparticles, have been crafted to recognize and to take advantage of localized acidosis that is a result of and therefore an indicator of many cancers.

Delivery devices have become more sophisticated and tinier progressing from milli-scale through micro-scale and now nano-scale. These new-generation nanotechnologies are now readily accessible to medical researchers and are expected to provide refined sensor and imaging abilities resulting in new possibilities in medicine, in particular, in mitochondrial medicine. By revolutionizing the ability to perform minimally-invasive, repeated, quantitative assessments of mitochondrial function in patients, new options for therapies can become available. It has long been known that whole body, blood, and tissue oxygen (O₂) levels are elevated in mitochondrial disease due to an impaired O₂ extraction efficiency when RC (OXPHOS) function is compromised. For example, a Clark-type O₂ nanosensor can, when integrated into a needle microarray, monitor in vivo mitochondrial function in selected tissues of patients with only minor discomfort and low risk to the patient.

This nanotechnology approach to in vivo O₂ measurement will be clinically feasible, well-tolerated, and sufficiently sensitive to enable reproducible and clinically meaningful objective measures of cellular and with particular respect to the present invention, mitochondrial function. Although not essential to practice of the present invention, an O₂ nanosensor that traverses the skin through a layer of micro-fabricated stainless steel (SS) needles (e.g., ˜25 μm thickness) to measure intra-organ O₂ levels may be employed as an indicator of mitochondrial function.

While most of the anti-cancer strategies discussed herein are most acceptable when concentrated at tumor sites or within cancer cells, general treatments such as food supplements also have a place in treatment and prevention of cancer. One characteristic of cancer cells (and super-proliferative cells in early stages of cancer pathways) is decreased OXPHOS and increased reliance on cellular glycosylation.

During fetal development, i.e., a period of life predominated by adding new cells, a large proportion of cells contribute to development of new organs or features and growth in general by forming additional cells, i.e., undergoing mitosis. In the adult, most cells have terminally differentiated to take on special tasks such as nerve cells, skin cells, liver cells, etc. These terminally differentiated cells lose the ability to divide. But in each organ a population of cells called stem cells remains less differentiated and maintains ability to divide. Usually the stem cell divides in an asymmetric fashion producing: i) one task driven tissue specific terminally differentiated cell that is incapable of further proliferation and ii) another stem cell. So in the body, not only cancer cells, but other cells are capable of dividing. One commonality observed in dividing cells is a de-emphasis on OXPHOS through the ETC and a greater reliance on glycolysis.

The present invention can facilitate the body's natural devices to control unneeded cell proliferation by supporting mitochondrial metabolism. To slow counterproductive cell proliferation and therefore development of cancers, support of OXPHOS by maintaining healthy mitochondria will shift the proliferation associated glycolysis weighted energy balance towards greater reliance on OXPHOS and thus make cells less prone to division. Restricting caloric intake can force an organism to be more efficient in energy (ATP) production and thus guide the cell towards increased use of the mitochondria's Electron Transport Chains' OXPHOS pathways with less emphasis on glycolysis. Restricting caloric intake is known to decrease cancer incidence. It is suspected, but not proven that shifting the metabolic energy balance more towards much more efficient OXPHOS inhibits cell division and thus may be useful in slowing cancers' progressions. The present invention in several suggested rounds of therapy includes emphasizing OXPHOS and/or de-emphasizing glycolysis.

Several therapeutic compounds may be delivered as whole body supplements or may be targeted at cancer cells. For example, resveratrol at a dose of 50-500 mg per day, including, but not limited to: about 50 mg, 75, 100, 125, 150, 175, 200, and 250 mg per day can be delivered as a supplement to boost or support functioning mitochondria and their oxidative phosphorylation processes. Resveratrol has also been reported to suppress inflammation through lipopolysaccharide induced NF-κB-dependent COX-2 activation.

Cationic amino acid helices preferentially bind to the mitochondrial inner membrane due to the mitochondrion's extreme membrane potential. This binding can disrupt the membrane integrity leading to swelling and possible rupture of the organelle. Swelling itself tends to promote apoptosis. Chimerizing these helices to a finder sequence such as an antibody fragment like sequence, a viral receptor sequence, an angioreceptor recognizing sequence or the like that recognizes cancer cells or regions harboring cancer cells can target these cells for apoptosis.

Coenzyme Q10 (CoQ10) can also be supplemented in an organism's diet. CoQ10 is a participant in the Electron Transport Chain activity and acts to support and stimulate oxidative phosphorylation. Delivering CoQ10 in conjunction with other features of the present invention may be used to support a phase of mitochondrial activity with resulting induction of apoptosis and/or inhibition of cell proliferation/division.

Coenzyme A (CoA) is especially important for delivering fatty acids to the mitochondrial outer membrane where carnitine palmitoyltransferase 1 exchanges acetyl CoA for carnitine. The reverse exchange occurs inside the mitochondrial inner membrane under the influence of carnitine palmitoyltransferase 2. CoA is synthesized by mitochondrial outer membranes in response to reduced caloric intake. This appears to be one of the compensating responses linking increased ETC and OXPHOS activity to reduced nutrient availability. Supporting CoA activity and its interface with 1-carnitine can help shift metabolic balance from glycolysis towards OXPHOS. Pantothenic acid or pantothenate, the acid counter ion, is found in vitamin supplements containing vitamin B5. Vitamin B5 is a precursor of CoA with pantotheine as one of the intermediate compounds. A dimer of pantotheine, pantothine, is an effective means for delivering pantotheine to the body's cells. CoA is not just required for transporting fatty acids to mitochondria but supplies acetyl groups to other enzymes for inactivating or activating genes. B5 shifts the ATP production away from glycolysis and towards the mitochondrial OXPHOS pathway.

L-carnitine is another glutathione stimulant capable of increasing ETC activity within mitochondria. L-carnitine also assists transport of fatty acids across mitochondrial membranes by replacing CoA as a fatty acid carrier to transport the molecules to the mitochondrion interior for metabolism. Acetyl-l-carnitine is a preferred compound for oral delivery of l-carnitine as it is more efficiently absorbed in the small intestine.

Supplemented acetyl-l-carnitine has been shown to attenuate mitochondrial fission. As related above, cancer cells' mitochondria have elevated fission with respect to fusion. By favoring OXPHOS over glycolysis, interfering with mitochondrial fission, and stimulating glutathione, metabolic shifts associated with neoplastic activity are reversed.

Alpha lipoic acid (or α-lipoic acid) stimulates burning sugar and fatty acids using oxidative phosphorylation. Alpha lipoic acid stimulates glutathione activity within cells and has widespread effects within cells including increasing mitochondrial function. This dual boosting effect on mitochondria shifts cells towards simple growth development and maintenance and inhibits proliferative activity.

Selenium is a metallic cofactor important for enzymatic function for such enzymes as the glutathione peroxidases. Selenium inhibits mitochondrial fission and thereby shifts the fusion/fission balance in favor of non-proliferation of the cell. Reduced fission is one factor relating to facilitated apoptosis of the cancer cells—so selenium also supports the cell's initiation of apoptosis initiated cell death. Oxidized glutathione promotes the oligomerization of the fusion proteins Mfn1, Mfn2 and Opa1 to activate fusion further shifting the fission/fusion balance in the direction against that of proliferating cells.

Control of levels of Opa1, the inner membrane fusion protein appears necessary to maintain fused mitochondria. When the amount is greatly elevated or depressed, transient membrane fusion activities occur, but complete fusions disappear. Since the mitochondrion has two membranes, complete fusion requires an initial fusion stage involving the outer membrane. Mfn1 and Mfn2 are anchored on the outer membrane and guide the fusion process there. OPA1 resides in the inner membrane. These fusion proteins bring membranes together by forming interlocking coils and, using GTP as an energy source, drive combination of the membranes. Since fusion has an anti-fission, anti-oncolytic effect it is interesting to note the correlation of obesity with cancer and the observation that obesity correlates with reduced Mfn2 expression. Many cancer cells have diminished Opa1 expression. Remedying this is one means for maintaining larger fused mitochondria in the mitochondrial network.

Repressing Mfn2 causes morphologic and functional breakdown of the mitochondria network through fission. And significantly, reduced Mfn2 availability inhibits glucose oxidation, reduces mitochondrial membrane potential and total cell respiration, while increasing mitochondrial proton (H⁺) leak.

Thus Mfn2 expression supporting maintenance of the fused mitochondria in the network is important to mitochondrial metabolism, including OXPHOS, and a properly functioning cell.

In an opposite activity, Drp1, a protein encoded by nDNA and found in the cytoplasm, when phosphorylated at a particular ser residue (S637) combines with Mff and fis1 to fragment the membrane. Mdivi1 inhibits Drp1 fission initiation by preventing the necessary phosphorylation. Supporting Mdivi1 through increased translation and/or expression is one tool for maintaining fused networks.

The size of the mitochondrial network at any given moment arises from the combination of mitochondrial biogenesis (creation of new mitochondrial material) and mitophagy (mitochondrial autophagy, which degrades mitochondria). These processes can respond to the needs of the cell. The increase in both the mitochondrial protein content and the physical size of the mitochondrial network when yeast cells transition from non-respiratory to respiratory conditions is an example of the upregulation of biogenesis to generate increased mitochondrial content. On the other hand, mitophagy is induced when cells experience a variety of stresses. For example, growth of yeast cells in nitrogen-depleted media induces both general autophagy and mitophagy to generate nitrogen for essential cellular processes. Biogenesis and mitophagy have to be regulated to maintain the proper mitochondrial content during normal cell growth.

ATP production by mitochondria requires nicotinamide adenine dinucleotide (NAD). Several studies including, but not limited to: J Biol Chem. 2004; Cell Metab. 2011; Mol Pharmacol., have demonstrated that NAD levels are OXPHOS limiting. The importance of NAD may be understood from its availability from at least four different synthesis pathways. Tryptophan, nicotinamide, nicotinic acid and nicotiamide riboside. Nicotinamide and nicotinic acid are forms of vitamin B₃ and can be delivered orally. Tryptophan is an amino acid and therefore is provided in a protein rich diet. Supplementation with these facilitators or mitochondrial ATP and transmembrane proton gradient opposes glycolysis and thereby favors non-proliferation attributes of the cell.

Dichloroacetate (DCA) (a minor contaminant resulting from chlorination of drinking water) is another strong potentiator of apoptosis. DCA is known to disrupt mitochondrial membranes allowing protons and cytochrome c escape into the cytoplasm. DCA also inhibits synthesis of pyruvate dehydrogenase, an enzyme essential to the glycolytic pathway which proliferating cells favor for ATP production. This forced shift of glycolytic/OXPHOS balance in the direction of non-proliferation slows production of new cells and also facilitates apoptotic activities which remove unnecessary or diseased cells. The result of DCA supplementation of cells already slanted towards apoptosis by other means is a stronger drive to initiate apoptosis in the cell.

Omega 3 fats, commonly present in fish oil, can also be used to shift the glycolysis/OXPHOS balance in the direction unfavorable to proliferation.

Flavones or flavonoids, for example, 3,3′,4′,5,7-pentahydroxyflavone.2H₂O, 2-phenyl-4H-1-benzopyran-4one, etc. are purified natural plant products or derivatives of natural plant products. Flavones may be supplemented through a diet emphasizing flavone or flavenoid containing fruits and/or vegetables. They are classified by several nomenclatures or groups including, but not limited to: anthocyanins, procyanidins, flavanones, flavones isoflavones, flavonols, flavon-3-ols, etc. Many flavonoid supplements are available commercially in varying degrees of purity from, for example, simply fresh or dried fruit, plant extracts to purified chemical compounds. These supplements may be anti-apoptotic in the sense they have anti-oxidant characteristics. But, for example, a flavenoid like 3,3′,4′,5,7-pentahydroxyflavone may be incorporated into one or more compositions as part of this invention because of it action to inhibit mitochondrial ATPases and thus favor apoptosis. Flavones are reported to increase uptake of lactate into mitochondria. Flavones are associated with an increased production of mitochondrial superoxide anions and concomitant apoptotic cell death. In addition to apoptosis induction flavones are involved with cell cycle arrest, caspase activation and inhibition of tumor cell proliferation.

One mechanism of flavone/flavenoid activity is especially relevant with respect to cancer cells. Lactate is a co-end product obtained when glycolysis produces ATP. Since cancer cells favor the glycolytic pathway over the more efficient mitochondrial ETC, flavone facilitated delivery to mitochondria of the lactate produced by the cancer cell's glycolysis shifted metabolism, increases generation of mitochondrial O₂⁻* radicals (unstable or reactive O₂ with an extra (unpaired) electron) which shifts the cell towards an apoptotic event. Thus supplemented flavone shifts the predominantly glycolytic metabolic pathway of neoplastic cells towards the more ETC based metabolism of normal cells. Flavones also arrest cell proliferation (division/mitosis) by halting progression from G0 to G1 phases of mitosis. 3,3′,4′,5,7-pentahydroxyflavone is also reported to activate deacetylase SIRT1 which also supports apoptotic processes. Flavones have been observed to reduce membrane potential and ion fluxes and permeabilities which may further contribute to their cell death promoting effects.

2′,3,4′,5,7-pentahydroxyflavone is another flavenoid discussed herein as an example. Like other flavenoids it has anti-oxidant effect, but also supports lipid peroxidation. It also induces apoptosis and interferes with proliferation by arresting mitosis at the G2/M phase interface. It is reported to have endonuclease activity and to suppress NF-κB activation which has both anti-cancer and pro-cancer properties. NF-κB is a potent inflammatory cytokine the body elicits against some existing neoplasms, but its inflammatory results are associated with initiation of some cancers. Thus NF-κB suppression is one means for reducing chemical damage to cells that if not properly mitigated can progress to cancerous lesions.

Thyroid hormone at elevated concentration and pharmaceutical mimics of the hormone can result in decreased mitochondrial membrane potential and through this effect and general metabolic stimulus increase the production of apoptosis promoting ROS.

Another natural factor that can be beneficially manipulated is the biologic membrane, for example, a class of membrane components called ceramides. Ceramides are an interesting group of compounds found chiefly in biologic membrane bilayer structure. They are amphiphilic molecules that are integral to the lipid bilayer structure of membranes, but when liberated to aqueous phase can act as intercellular and intracellular signal molecules. Ceramides have been recognized as favoring mitochondrial fission. Since mitochondrial fission acts as a brake on apoptosis, inhibiting ceramide fissile activity can potentiate apoptosis by restoring the fusion/fission balance to more normal levels and thereby potentiate apoptosis of ceramide inhibited cells. Fumosins, natural mycotoxins frequently found in grain stores, and fumosin analogues are particularly effective in this endeavor. Using natural mycotoxins or synthetic mycotoxin like structures, by favoring fused mitochondria, can also remove some of the blockages to apoptosis that cancerous cells might have developed which act to impede natural or the therapeutic anti-cancer effects of one or more other constituents in a cocktail provided by the end goals of this invention.

The mitochondrion has two membranes which maintain pH gradients—with the inter-membrane space being relatively acidic to both the mitochondrial matrix the innermost and most basic mitochondrial compartment and the cytosol outside the mitochondrial outer membrane. Drugs that are permeable through biologic membranes may distribute based on charge with their charges determined by protonation state which varies with pH. Several compounds obtain greatly enhanced activity depending on pH. For example, transition or rare earth elements, with multiple oxidation states display pH sensitivity. Gadolinium is one such element whose toxicity may approach lethal levels as the pH decreases but is much less toxic in regions of higher pH. Incorporating one of these ions or one of the several peptides that also increase toxicity at low pH into a carrier molecule or particle, e.g., a membrane crossing peptide, a lipoprotein, a liposome, a nanoparticle, can effect entry into targeted cells to produce desired toxic affect.

When membrane permeability is increased by activation or opening of the mitochondrial permeability transition pores (MPTP), the pH gradient is destroyed as ions up to a size about 1.5 kilodalton are free to diffuse through the open pores. Hydrogen ions being especially small (just a single proton, 0.001 kilodaltons) transgress rapidly through the openings and destroy the pH gradients. MPTP activation has several pathways including, but not limited to: accumulated Ca⁺⁺ in mitochondria, increased Ca⁺⁺ flux, inhibited Ca⁺⁺ATPase, ROS, increased ER Ca++, diminished transmembrane potential, pro-oxidants, etc. MPTP activation effects apoptosis through several potential mechanisms. Since the H⁺ gradient provides the energy for ATP production, destruction of the H⁺ gradient by opening MPTPs (or by other means) results in rapid ATP depletion. The lack of ATP has widespread effects, a major one being that ion pumps on the plasma membrane (the membrane encasing the cell's cytosol) cease functioning. For example, Na⁺/K⁺ ATPase and Ca⁺⁺ ATPase no longer maintain the ion gradients. This leads to cell membrane depolarization. Ca⁺⁺ ions rapidly accumulate in the cytoplasm and cause cell death through necrosis. Alternatively, cell death through apoptosis can occur when mitochondrial MPTP permeability allows release of cytochrome c and apoptosis related peptides including caspases and apoptosis inducing factor (AIF) into the cytoplasm. When anti-apoptosis defenses are insufficient to counteract these apoptosis inducing events, the cell will die a controlled apoptotic death. Betulinic acid, arsenite, CD437, several amphiphilic cationic α-helical peptides, etoposide, doxorubicin, 1-β-d-arabinofuranosylcytosine and ionidamine can use MPTP to shift the cell towards apoptosis.

Reactive oxygen species as a class of compounds are known to induce apoptosis. Ultraviolet or ionizing radiation, transition metal ions and some xenobiotics are methods that have been used to increase ROS and to tilt the balance towards apoptosis. Cis-1-hydroxy-4-(1-naphthyl)-6-octylpiperidine-2-one, by increasing production of damaging active oxygens, can contribute to or may induce apoptosis. Shifting metabolism from the ETC oxidation pathway towards glycolysis is one means of reducing ROS production and thus deemphasize one apoptotic stimulus. Conversely, emphasizing the OXPHOS mechanism can reverse this anti-apoptotic tilt. AZT a therapeutic compound use to treat acquired immune deficiency virus infection exhibits cellular toxicity in part through increasing ROS production.

The MPTP resides in the inner mitochondrial membrane and does not directly destroy the outer mitochondrial membrane permeability barrier. But the opening of the pore allows a massive flux of particles into the inter-membrane space. As these particles move, water follows the osmotic gradient to cause massive swelling and subsequent mechanical rupture of the mitochondrial outer membrane. Apoptosis initiating cytochrome c and other pro-apoptotic proteins are thereby released into the cytoplasmic space where the disintegration processes of apoptosis can then transpire.

Still another path is available for mitochondrial compromise to induce apoptosis. Pro-apoptotic proteins, Bak and Bax, can associate in the outer membrane to provide outer membrane permeabilization. As cells normally function, pro-apoptotic Bak and Bax do not associate to cause cell death. Anti-apoptotic influences must be stronger if a cell is to function. Removing stabilizing anti-Bak/Bax influences would tilt the balance towards apoptosis of the affected cells. Accordingly, one aspect of the invention may include modifying expression of anti-apoptotic proteins including, but not limited to: Bcl-xL and Mcl1 that inhibit Bak/Bax permeabilization of the mitochondrial outer membrane. Methods such as RNAi and gene editing, for example, using a method like CRISPR, would be effective. For example, when a virus is used to target cancer cells, the virus can include such expression suppressors.

Tumor Necrosis Factor alpha (TNF-α) induces apoptosis through support of Bak/Bax linked permeabilization of the mitochondrial outer membrane. However, since TNF-α can activate both pro-apoptotic and anti-apoptotic pathways, it is helpful to determine which is the dominant effect in the targeted cell before when this strategy is considered.

Cell surface receptors associated with initiating apoptosis pathways can also be used to tilt the balance in favor of apoptosis. For example, expression and incorporation of Fas into the plasma membrane can augment apoptosis. For example, genetic engineering to facilitate transcription or translation is an elegant tool to achieve this. Ceramides are believed to stimulate expression of Fas into the cell's plasma membrane. Any compound, for example, daunorubicin and the like, that increase ceramide activity may stimulate apoptosis through this path.

Depending on cell type and specific cancer inducing or cancer evolved mutations one or another of these cell deaths pathways may be accelerated at different stages of therapy when multiple cocktails are prepared for sequential therapy. Another factor to consider is other treatments the subject may have received or be receiving. For example, COX2 inhibitors at high doses may promote mitochondrial swelling and compound apoptotic influences, but their possible decoupling effect, at some concentrations, may oppose apoptosis. N-acylethanol-amines at high concentrations can reduce mitochondrial membrane potential thereby favoring apoptosis, but at lower concentrations have an effect of closing MPTP with an associated anti-apoptotic tendency.

The invention may have increased desired effect if multiple modes are practiced sequentially. Neoplastic cells are known to change their characteristics during the disease process. These cell lines may evolve in response to the body's defenses successfully eliminating some cells. Survivors will have developed characteristics allowing survival in the face of the body's defenses. Similarly, treatment if not 100% successful in eliminating all neoplastic cells will leave survivors with survival characteristics differing from the dead cells. Accordingly, a particularly robust embodiment of the present invention features multiple therapeutic interventions on a schedule that changes as the neoplastic cells are expected to mutate for their survival.

Adoptive T cells, T cells cloned with a tumor specific antigen receptor, have been partially effective in fighting cancer. T cells are cultured in the presence of tumor cells and those most reactive to the tumor cell surface proteins are cloned. One or more of these clones was then re-infused into the patient to initiate a T-cell driven immune response.

A variant of this method identifies the antigen receptor on the T cell and further identifies the binding portion of the receptor. A stabilized receptor (binding fragment) is engineered for insertion into a targeting moiety. The moiety may be completely synthetic, such as a liposome with receptor embedded in its bilayer or may be a modified biological derivative, such as an enucleated cell transporting antiproliferative therapeutics to cancer cells, a biologic body without a nucleus (e.g., an inside out red cell, modified platelet, etc.), a modified virus, a modified immune cell etc.

Several, but unfortunately not all, cancers feature generously hyper-expressed surface proteins or enzymes whose activity can be readily targeted using binding ligands. Biopsies and screening, e.g., protein chip, cDNA analysis, etc. may be used as tools to identify these features for targeting therapies or sometimes for simply assessing progression of cancer or the treatment. However, it should be considered that not all cells will express the same genes. The age of the cell, interaction with neighbor cells, status of mitochondria, availability of a blood supply, etc. may result in differentiation of surface characteristics. The present invention by continuously altering therapeutic approaches explicitly recognizes this likelihood.

However, one commonality of virtually all cancers is the depressed pH in their vicinity apparently the result of the glycolytic production of lactic acid. The metabolism common to cancer cells results in extraordinary H⁺ production. PET (positive emission tomography) largely confirms this.

Another way that substances can be specifically targeted to a biologic entity uses tools that biology itself has evolved for us. Viruses are relatively simple biologic entities having essentially only genetic material and a packaging material. The virus infects a cell and co-opts its synthetic and metabolic functions to reproduce viral particles. Attacked cells frequently fight viral infection by a suicide response such as apoptosis. To counter this many viruses have developed means to avoid apoptosis.

Viruses may also benefit from causing apoptosis, for example, to act against immune cells attacking a virus or virally infected cell, or for breaking apart the cell to spread the virus. The present invention includes the apoptotic stimulating abilities of viruses. But virus genomes are readily engineered, so a wild type virus with great anti-apoptotic influence may be altered to lose this capability or even to accelerate apoptotic initiation.

For example, a first therapeutic approach may employ an oncolytic virus such as reovirus to gain entry to and to infect cancer cells. Viral infection will trigger an immune response drawing Natural Killer cells and T-lymphocytes to the infected area. Reovirus has been in clinical trials in combination with commonly infused chemotherapeutic drugs such as gemcitabine, paclitaxel, carboplatin and docitaxel. These chemotherapeutic agents have been shown to prolong lives in many patients, but like many cancer drugs the anti-neoplastic effects wear off while the patient's healthy cells do not seem to invoke similar tolerance. Use of the reovirus as a treatment adjunct was attempted in order to enhance immune response.

Reovirus is one of a group of viruses that support their proliferation through avoiding activation of protein kinase R (PKR) in response to presence of double-stranded RNA within a cell. PKR has a double-stranded RNA binding site that a) inactivate the RNA and b) through phosphorylating other proteins. Reovirus and others like it take advantage of the characteristic of the majority of neoplastic cells wherein Ras is activated. The Ras oncogene prevents PKR activation by blocking its phosphorylation. As a result cancer cells, with mutated ras activation preferentially allow reovirus and other retroviruses normally defended against by PKR to survive and proliferate. This property makes retroviruses such as reovirus preferentially effective against cancer cells so long as the PKR activation remains faulty.

A retrovirus, such as a reovirus, thus becomes a means for preferentially accessing inner workings of cancer cells. While the reovirus is capable of proliferating within and eventually killing cells which it infects, the viruses, while preferentially killing cancer cells are insufficiently effective at killing a majority or larger fraction of non-cancerous cells populating the tumor zone and may be ineffective at killing all cancer cells.

Another example is HSV where the R3616 mutation causes the ICP34.5 protein to bind with protein phosphatase 1, another means of preventing PKR activation. Another example includes mutation of the UL39 gene (in herpes simplex) to enhance viral proliferation in cancer cells. These and other cancer specific targeting means can be used advantageously in the present invention.

Rather than relying on conventional extremely toxic chemotherapeutic drugs that attack needed healthy as well as the neoplastic cells, a more effective approach is to increase lethality of viral attack. In place of traditional toxic substances that generally interfere with mitosis (cell division) in rapidly dividing cancer cells as well as other normal rapidly dividing cells, like liver cells and those producing blood, proliferation may be halted by causing cancer cell death rather than simply reducing the number of new cells produced by actively dividing. For example, the virus can be supported by using components common to cells that augment the apoptosis pathways. Another means of augmenting apoptosis is to modify virus to carry pro-apoptotic elements into the infected cell. Site directed mutagenesis is one conventional tool for modifying viruses and other genomic material. Since viruses are rapid proliferators, random mutations can be used as a tool for making viruses with more desired properties. Selection of viruses with desired traits and their proliferation is also known in the art for making modified virus.

For example, the modified reovirus may deliver CRISPR like machinery targeting Bcl or another anti-apoptotic protein. Since neoplastic cells have multiple mutations in nuclear as well as mitochondrial DNA, and the mutated DNA are homoplasmic, identifying mitochondrial mutations even if the mutations appear silent with respect to gene product, can allow genetic material of these mutated mitochondrial DNA to be targeted specifically. In other words, if the virus delivers its modified genome inside a cell, material complementary to the mitochondrial mutation, the modified genomic material will not find a partner to bind with in the unmutated mitochondria. This will help protect non-neoplastic cells from mitochondrial disruption.

Accordingly, the therapeutic intervention may also include RNAi specific to a mutated mitochondrial gene within that neoplastic line. RNAi broadly describes a natural or induced biologic process wherein small RNA molecules inhibit or halt expression by interfering with action of an RNA with which the small RNA forms a complementary association. Small RNA may be introduced as self-acting molecules or may be manufactured following its coding sequence having been inserted into the host DNA.

A second intervention might use a differently modified reovirus, perhaps addressing expression other proteins, either by shut down, or mutation. A third therapeutic intervention might preferably employ a different virus family modified to infect tumor cells, but perhaps using a different recognition site on the cell, or a virus modified to incorporate a recognition partner from another virus or an antibody like portion that preferentially binds or recognizes a cancer cell surface protein.

The subsequent viral delivery vehicle may repeat previous intracellular action such as turning off one or more anti-apoptotic proteins and may also include, in its modified genome, additional components to compromise the surviving neoplastic cells, perhaps causing expression or an immunogenic protein on the cell surface; perhaps interfering with mitochondrial fission. The order of these changed therapies in not a required aspect of the sequential practicing of this invention. The therapies may overlap in time. The surviving cells would face dual stresses as a first therapy was replaced by a second. The important consideration is that multiple strategies are applied in sequence a the earlier therapies take their tolls on the cancer cells leaving in the tumor cells that have evolved resistance to the therapy originally effective on the original cell population. There is evidence that some tumor cells remain in a quiescent state, possibly because of their location and availability of stimulating growth factors or nutrients. Accordingly, repeating an earlier effective therapy that has seen diminished effectiveness may render a second or third round, etc. capable of effective results.

Repeating the screening indicating presence of, type of or degree of cancer may be used to guide therapeutic patterns.

In conjunction with the present invention other therapies are not contraindicated. If the patient and physician determine that, for example, a course of radiation would advantageously shrink the tumor or increase cell vulnerability, radiation treatments can continue in coordination with the therapies described in the present invention. In a big picture view, the radiation treatments might be seen in the same light as one of the serial therapeutic compositions.

Detection (identification) of cancer cells is accomplished by a variety of means. For example, U.S. Pat. No. 7,700,307 to Murray et al uses mitochondrial stress-70 protein to recognize colo-rectal cancerous cells. Although assays are available to monitor treatment effectiveness for many cancer types, targeting individual cells or groups of cells having sufficient placement and numbers of mutation events to render the cells cancerous is not satisfactorily available for significant numbers of cancer cell types. While many biologic markers are suitable for ex vivo analysis to monitor cancer presence or progression, additional means for targeting therapeutic interventions predominantly to cancerous or precancerous cells are desired.

With the advance in molecular medicine, human understanding of molecular biologic events underlying cancer initiation, progression and treatment has improved giving clinicians and patients better therapeutic approaches for managing or curing cancer. However, since a neoplastic cell is essentially a de-differentiated or partially de-differentiated cell with chromosome instability, repeated novel mutations occur, allowing a form of Darwinian selection of surviving tumor cells. This repeated morphing process has the effect of making initially used anti-cancer drugs obsolete for the newly selected surviving population of cells. Thus, new pharmaceutical compounds and treatment strategies that can circumvent complications of current therapeutic approaches are often required.

A novel concept has emerged whereby targeting of mitochondria has become increasingly recognized as a promising and highly effective anti-cancer approach Ralph and Neuzil (U.S. Pat. No. 8,598,145) have proposed the term, “mitocans”, referring to small molecules with anti-cancer activity that induce apoptosis by destabilizing mitochondria in cancer cells. Seven possible mitocans are discussed there. While this general approach has merit, the specificity of “small molecules” to attack cancer cells with specificity remains difficult for many cancers. Thus more robust and refined specific delivery and selected activation of treatment methods are still desired.

A measurable difference in cancer cell mitochondria compared to normal cell mitochondria is the greater mitochondrial inner trans-membrane potential in cancer cells' mitochondria. For example, as a result of the metabolic changes occurring inside cancer cells and their mitochondria, the transmembrane potential increases magnitude to greater negative values (about −150 to −170 mV, negative inside the matrix) in carcinoma cells. The mitochondrion actually has two membranes and thus electrochemical gradients across each to give a net mitochondrial potential between cytoplasm and matrix. The outer membrane is relatively porous due in part to its voltage dependent anion channel which allows polar molecules less than about 5 kilodaltons to cross. Specific proteinaceous carriers facilitate crossing by other molecules either by providing ion specific pores or using carrier proteins. The inner membrane is more impermeable allowing only small polar molecules like water or ammonia to cross. Nonpolar (lipophilic) molecules more easily cross these lipid bilayers without necessity for pores or specific carriers.

One additional therapy may include facilitating mitochondrial function. The neoplastic cells have evolved with mutated mitochondria. Restoring mitochondria to a more normal level of activities, especially including fission resistance, may provide additional stress on the cancer cell and may render these cells more prone to apoptosis. Systemically boosted mitochondrial activity, i.e., providing stimuli and nutrients to enhance energy production and synthesis of by-products of mitochondrial energetic functions may assist cells that may have started a path towards neoplastic growth to heal themselves, or enhanced mitochondrial may assist the organism's processes for eliminating the unhealthy cells.

Many hypotheses have been made to explain this difference in membrane potential. E.g., at the molecular level, these include differences in mitochondrial respiratory enzyme complexes, electron carriers, ATPase, ANT and/or changes in membrane lipid metabolism. Other suggestions for the increased mitochondrial transmembrane potential in cancer cells include altered electron transfer activity, proton translocation and utilization, or conductance. See e.g., U.S. Pat. No. 8,598,145 for more discussion.

Also significant changes in enzyme function, particularly in the ATPase, have been shown to occur in cancer cell mitochondria. Preparations of ATPase isolated from carcinomas show reduced maximal velocity, decreased immune-detectable levels of the (3-subunit of the F1 component of mitochondrial ATPase and/or overexpression of the ATPase inhibitor protein (IF1). A reduced ability to use the proton gradient to make ATP, with a resulting build up in the protons within the MIM would account for the greater transmembrane potential existing in tumor mitochondria. Another action that may account for greater transmembrane potential in cancer cells is that acetoin undergoes an ATP dependent reaction, almost doubling the reaction rate to produce citrate in tumor cells which is then exported by the tricarboxylate or citrate carrier (CIC) to the cytosol where it is cleaved to oxaloacetate and acetyl-coA. The net effect is a high level of cytoplasmic acetyl-coA precursor for sterol biosynthesis capable of promoting an otherwise elevated cancer cell production of cholesterol. The build-up of cholesterol in the inner MIM reduces several fold the passive proton permeability, thus forming the greater transmembrane potential in cancer cells.

Enhanced glycolytic activity from the cancer cells' high energetic demand increases cytoplasmic levels of lactic acid production in these cancer cells. To maintain the pseudo-neutral pH of the cytosol the cells activate plasma membrane proton pumps leading to extracellular acidification. Often, pH of the tumor interstitium is ˜6.2-6.5, while pH of non-cancerous analogous tissue interstitium is neutral, ˜7.0. The pH difference between tumor and normal tissue presents a lucrative tumor specific target for targeting and enabling treatment of cancer.

These observations led to development of a novel anti-cancer strategy by inhibiting the proton pumping activity of the ATPase, causing acidification of the cancer cell cytosol that, in turn, results in the demise of the cell. Small interfering RNA targeting to the subunit ATP6L of proton pump have been used to prevent the cancer cells' de-acidification, driving a still lower pH to kill cancer cells.

Cancer cells also undergo prolific growth and mitosis. Regardless of the cancer cell origination, all cancers, including, but not limited to: breast cancer, osteosarcoma, angiosarcoma, fibrosarcoma, leukemia, lymphoma, ovarian cancer, uretal cancer, bladder cancer, prostate cancer, genitourinary cancer, colon cancer, esophageal cancer, stomach cancer, gastrointestinal cancer, lung cancer, myelomas, pancreatic cancer, liver cancer, kidney cancer; endocrine cancer, skin cancer and brain cancer; share traits of reduced cell death and undesired proliferation (uncontrolled growth). The minimally controlled or apparently uncontrolled growth and proliferation of these cells results from mutation events within the nuclear DNA. The cell's mitochondria in response to the same stresses also experience numerous mutations. Several of these mutations are selected out through mitochondrial fusion and fission, but many remain, either in non-coding regions or in mutations that either are not seriously deleterious or, for example, in the lower pH confines, may in fact be beneficial. Mitochondria lack repair mechanisms found in the cell nucleus so mtDNA mutations are more stably maintained and therefore targetable by molecules for example polynucleotides made to interact with the mutated mtDNA.

Cancer cells express mutations that affect normal control of cell proliferation. The ras family of genes is involved in cell signaling. The Ras pathway is recognized as critical in cell proliferation; mutations along this pathway that maintain an active Ras pathway are essential to development of most if not all cancers. Cells with activated Ras have thus been considered as targets for anti-cancer therapies. Following this strategy a reovirus has been found that specifically recognizes and proliferates in cells with activated Ras pathways. The virus does not itself target cells containing ras, but will infect cells whose Ras pathways are activated by other non-ras mutations.

Reoviruses, as discussed above, are retroviruses, viruses whose genetic coding is made from RNA. To infect a cell and proliferate the virus, the RNA must enter the cell nucleus and be inserted into the host cell DNA using the reverse transcriptase enzyme. Safety concerns relating to infection from the therapeutic use of reovirus can be managed by genetically engineering the virus to prevent a portion, including, but not limited to: the viral capsid, from developing.

Reovirus has been used to infect neoplasms (whose cells are believed to be Ras active cancer cells) and to lyse these cells thereby reducing tumor size. However, complete tumor destruction is not commonly observed. Since viruses are essentially small packages of genetic material they are easy to engineer, for example, to disable one gene of the virus, or to insert additional genetic material. Retroviruses with the ability to insert their genome into the host cell nuclear DNA are suitable carriers for genetic or other material into a cell and cell nucleus.

In the context of cancer therapy, the recognized strengths of reovirus in infecting mainly active cancer cells and killing many, can be improved by incorporating baggage inside the virus that can elicit alternative means of killing the infected cell. A modified reovirus or other virus selected or engineered to gain entry into cancer cells can carry elements lethal to the entered cell or in a refined example carry elements whose lethality is targeted at a cancer cell specific part or cancer cell specific molecule potentially in the infected cell. This refinement adds an additional safety measure, requiring two cancer related events to be present thereby sparing Ras activated or other non-cancerous cells the virus may have entered.

Strains of vaccinia virus, herpes virus, vesicular stomatitis virus, senaca virus, Semliki Forest virus, ECHO or REGVIR virus, and monstrously attenuated polio virus have been similarly tested and characterized in cancer cells or in animals or humans with cancers for their inherent cell killing effects primarily targeted at cancers.

Bcl-2 is an important anti-apoptotic protein. Several factors such as galectin-3 and similar lectins are supportive in this activity. Suppressing anti-apoptotic paths in the cells targeted for destruction may synergize the therapeutic efforts of the present invention. Mitophagy defects are known in disease. For example, pulmonary fibrosis has been shown to involve apoptosis resistance with a relation to mitophagic progression in alveolar cells. The dual character of mitophagy as initiator or protector with respect to apoptosis is complex.

One feature common to many cancers is dysregulation of apoptosis. Bcl2, an anti-apoptotic protein, is often hyper-expressed in cancer cells. The excess Bcl2 stymies apoptotic initiating action of cytochrome c and other pro-apoptotic releases (including, but not limited to: AIF, Endo G, HtrA2/Oma, Snac/DIABLO, Hsp60/10 and procaspase) from mitochondria and thereby aids cancer cell survivability. An optional element of the present invention addresses this cancer cell feature by addressing Bcl2 content in the cancer cell and/or by circumventing Bcl2 protective effects by activating one or more of the caspases.

Mitophagy may be a stepping stone to cancer cell apoptosis. Normally mitochondria are dynamic organelles. Mitochondria are constantly active as the energy centers of the cell. They change shape as needed. They translocate to high demand areas within the cell. Fusion and fission combine and split off new mitochondria. At times dysfunctional mitochondria self destruct (through a process called mitophagy or autophagy) saving their contents to be recycled within the cell. Mitophagy is the destruction of a mitochondrion through engulfment inside an autophagosome, an intracellular vesicle that has evolved to remove and recycle organelles and macromolecules that the cell no longer considers useful. Autophagy is the generic process. Mitophagy is a name used when autophagy destroys a mitochondrion. Mitophagy is designed to preserve the cell by removing a “bad” mitochondrion. But mitophagy can also participate in or perhaps elicit destruction of the entire cell through a process termed apoptosis.

Angiogenic factors are important for growth of tumors exceeding about 1 mm in diameter. Unless the tumor cells can encourage growth of blood supply, nutrients will not be available in sufficient quantities and waste products will accumulate poisoning the stressed cancer cells and their surroundings. Vascular Endothelial Growth Factor (VEGF) is a family of peptide hormones that contribute to vascular growth. Related proteins, such as VEGFRs (VEGF receptors), PIGF (which binds VEGFR1 and neuropilin1) and growth factor such as FGF, are labile factors that can stimulate endothelial cells to effect angiogenesis.

A tumor biopsy or blood sample can be used to identify angiogenic and other growth factors produces by tumor cells. When biopsied samples are small in comparison to the tissue of interest, biopsies from several portions of the cancer afflicted tissue may be advantageous as different stresses on different cancer cells may amplify one or more secreted factors.

Identification of secreted factors, e.g., growth factors, enables several branches of therapies. Growth factor inhibitors specific to those identified might be included as part of therapy. The growth or other secreted factor can also serve as a signal beacon to guide delivery of therapeutic compounds. Modified immune recognition proteins such as antibodies or antigen recognizing parts thereof or modified receptors for the factor can concentrate a therapeutic compound at the site secreting the factor(s).

Conversely, destroying an activity, perhaps preventing mitophagy, or mitochondrial fission may confuse the cytosolic cell sufficiently to cause apoptosis.

Mitochondrial involvement in cancer initiation and/or progression has been studied for over six decades. Given the differences in energy metabolism between normal and cancer cells, the energy organelle within the cell was clearly implicated as an important factor in transformed cancer cells. Additional energy requirements could be met by increasing mitochondrial activity or by increasing mitochondrial density. Changes in mtDNA content, changed expression levels and activity of the ETC or one or more of its subunits, and mutations to mitochondrial DNA mtDNA).

Fortuitously most mtDNA mutations appear to be homeoplasmic; all mitochondria in the cell appear to share the same mutation(s). This makes sense if one considers that tumors develop after several mutations in a cell lead uncontrolled division of the cell to form the tumor mass. One consequence of the homeoplasmic nature of mtDNA mutations is that the same effect can be achieved in all mitochondria of the cell. The worry that attacking mutated mtDNA might only remove faulty mitochondria while preserving unmutated mitochondria that might be especially useful for cell survival is actually not a concern.

Accordingly, one aspect of the present invention involves attacking mutated mtDNA. By focusing on the mutated mtDNA, effects will be limited to the cancer cells wherein the mtDNA is found. Mutations in the d-loop are especially accessible while the mtDNA genome is duplicating. This offers an opportunity to prevent the cell from adding additional respiratory capacity. Mutations in rRNA, and the various proteins are most accessible during translation. This offers an opportunity to permanently shut down the gene, for example, by cleaving, by covalently binding or by using a gene altering process such as CRISPR. Products of transcription, mRNA, may also be targeted using such tools as any form of RNAi.

Identifying the mutation(s) in the cancer mitochondria can be accomplished by sequencing mtDNA of biopsied tumor cells. The human mt genome has been sequenced with various insights relating to recent or ancient genealogy. These sequences may be used as comparators to identify mutations in the cancer cells. For a more robust comparison, the mtDNA from non-cancerous cells from the same individual, preferably even a biopsy of normal tissue close to the tumor. Once mutations have been identified anti-nucleic acid therapeutic molecules specific to the mutated sequence can be synthesized and evaluated. One option is to test these potential modes of attack on cells grown ex vivo from the tumor, possibly checking safety against non-cancerous cells.

The therapeutic molecules might be injected directly into a solid tumor, but delivery through circulation is advantageous since circulation can deliver to tumors that may not have been detected or visualized, or that may be difficult to reach.

One proven tool for targeting tumors and delivering cargo thereto involves using one or more of the cancer targeting or cancer specific viruses. The virus itself may preferably compromise the cancer cell(s) to a degree, but in conjunction with the attack on the cancer's energy source, much greater deleterious effect may be had. This added compromise of cell function in addition to any the carrier virus is causing will improve efficiency of the viral attack on the cancer and improve patient outlook.

When the mtDNA has mutated genes of the ETC, specifically targeting these mutated, but functional genes may be used to shut down the cell's energy production by interfering with the electron transport chain's ability to form the proton gradient across the mt membrane and thereby close off production of ATP. Mutated mtDNA may also provides means for targeting the H⁺ gradient that is necessary for generating the cell's ATP.

While preferably a cancer targeting virus is used for delivery of a therapeutic molecule specific to a mtDNA mutation within the cancer cells, less rigorously targeted (more generic) cargo can be transported by the virus. For example, decreasing cytochrome c oxidase (even absent a relevant mutation) may increase cytochrome c in the cytosol sufficient to elicit apoptosis. Or the carrier may transport compounds leading to interruption of the electron transport chain or uncouple the ETC from ATP production.

Another approach a clinician may desire is to accelerate rather than shut off the cancer cells metabolism. The mutated genes can be targeted to improve stability and to prevent degradation of the RNA to maintain or increase activity of the respiratory proteins. The increased respiration would have at least three effects deleterious to cancer cell survival. On the one hand, the increased activity would scavenge needed nutrients and since many tumor cells have barely adequate blood supply, starve the region of cells of the nutrients consumed and poison the cells with metabolic byproduct. A second deleterious effect would be that the additional chemical activity would produce local heat further stressing the specifically affected cancer cells and its neighbors that may not have received the same high delivery of therapeutic substance carried by the virus. The stress may not be tied to any one affected cell but may be shared with other cells in the vicinity. A third deleterious effect is that the accelerated metabolism may cause necrosis and thereby invoke an immune response against the cancer cells.

An increase in metabolism will because of the increased chemical activity generate an abnormal level of heat. MRI is able to remotely monitor (measure) temperature because the Larmor frequency of water protons demonstrates a linear temperature dependence. Temperature can also be monitored using internal probes, preferably probes whose radio frequency sensitivity has a measurable shift around the body temperature. MRI may be used as a guide for delivering the desired probe, preferably a small nano-scale probe that may detect temperature and/or one or more additional characteristics of interest.

The glycolytic metabolic branch that predominates in cancer and pre-cancer cells can be sensed by biologic or electronic sensors externally or the extraordinary heat output locally. These cells often will adapt to the local change in temperature by modifying membrane fluidity to approximate normal fluidity and functions. In addition to the external signatures of metabolic change these adaptations cause, these membrane changes can be used internally as signals for attracting or attaching probes, vesicles or small molecule binders. The glycolysis based metabolism will also produce chemical metabolites, e.g., lactate and H⁺ that can also be locally detected using probes. MRI is capable of detecting tumor related or pre-cancerous metabolites including, but not limited to: various amino acids, lipids, choline, creatine, myoinositol, and lactate.

One aspect of the present invention therefore features treating to prevent evolution of pre-cancerous cells down their paths to uncontrolled proliferation characteristic of cancer.

Cancer cells, like all living cells, require nutrients to support their metabolism and to grow. As they metabolize, waste products are produced. In larger multi-cell organisms, a circulatory system delivers nutrients, often including a fuel substance like fat or carbohydrate and O₂, to cells. The circulatory system removes product of metabolism that are not needed by the cell. The circulatory system relies on diffusion, pseudo random movement of substances from a higher concentration to a lower concentration zone. Permeability barriers may influence local diffusion. But diffusion is in essence a physical process depending on random movement of molecules and its effectiveness declines with diffusion distance.

Accordingly cells a farther than about 1 millimeter from a blood vessel face nutrient stress and waste material build-up. These cells do not function well, but a system has evolved that under nutrient stress the cells send out a signal to invite a blood vessel to from nearby. The process of adding additional blood vessels has been named “angiogenesis”. Angiogenesis is essential to support bulk growth (increased volume of tissue) or to provide healing after injury. As cancer cells form tumors, the cells in positions distant from the blood vessels will come under stress. Cells will adapt their metabolisms, some adaptations will include further mutations of nuclear of organelle DNA, many will die, but others will survive because of angiogenesis.

Angiogenesis is thus essential for maintaining a viable solid tumor. The necessity for angiogenesis provides several opportunities for anti-cancer treatments. Obviously, halting angiogenesis would halt tumor growth. However, tumor cells already with sufficient blood supply could continue to survive and these tumors could still bud off cells and form many more small metastatic tumors. Halting angiogenesis, would also interfere with the organism's healing processes, making aging more severe and rendering the organism more sensitive to injuries.

A second approach would be to slow or halt angiogenesis locally, i.e., to prevent the tumor cells from obtaining needed blood supply. Tumor cells might be modified to lose ability to secrete angiogenic signals. Secretion of proteins can be prevented at any stage from sensing a need for the secreted substance to releasing the signal to extracellular space. To promote angiogenesis when the cell senses oxidative, nutrient or metabolic stress, a transcription factor in the nucleus must bind to DNA to begin the process of transcription editing RNA, pairing messenger RNA with tRNA, transporting RNA to ribosomes, synthesizing a polypeptide at the ribosome, editing the peptide, folding the peptide, transporting to and secreting the peptide through the cell membrane. Blocking any point in the path could sentence the cell to nutrient starvation or metabolic poisoning. Local blood vessel stem cells, angioblasts, could also be locally inhibited, either by slowing or blocking recruitment of angioblasts or interfering with differentiation. These metabolic progressions would change the body's chemistry. Thus external probes similar to those employed in the cancer cell screening may be used to monitor cancer progression and/or cancer treatment.

A third approach would be to respond to the angiogenic signal from the tumor. An anti tumor substance is thereby concentrated near the stressed out tumor. Physical, chemical or biologic means or combinations thereof are options to be used to target and/or to attack tumors, tumor cells or parts thereof, including, but not limited to: cell membrane, cell cytoskeleton, ER, lysosome, golgi, peroxisome, nuclear membrane, nucleus, centriole and mitochondrion.

The present invention features several aspects including a method using associated compounds that destroys or weakens cancer cells by specifically interacting with the cell preferably through recognition of a mutated mitochondrion sequence. A mission underlying the ultimate goal of the present invention is to interfere with the mitochondrial activity required for cell survival and therethrough to lead to cancer cell death. The compound may be in an isolated, purified, substantially purified, synthetic or recombinant form.

The invention includes a variety of tools used individually or in combination. For example, a therapeutic composition may include several compounds and may further include physical as well as chemical/biological interventions. Therapies may include a course of treatment using composition A, an optional reassessment of surviving cancer cells including any adaptations the surviving cells may have made, a modified or second phase composition, another optional reassessment, etc. Any one approach or any combination of approaches parallel or in series are considered as features of the present invention.

The therapy may involve a plurality of therapeutic compounds, perhaps a collection of mutation targeting entities and optionally further including an antibiotic (antibacterial like substance, e.g., those like: bacitracin, gramicidin, enniatin, valinomycin, serratomolide, polymyxin, monomycin, alamethicin, beauvericin) that is particularly effective against mitochondrial function, proliferation, fusion or fission. Other optional therapeutics might be, those affecting mitochondrial porosity, delivery of mitochondrial proteins from the ER, free radical amplifiers, or osmotic swelling agents.

Any suitable type of delivery moiety may be used. For example, U.S. Pat. No. 9,402,839 to Wipf et al claims a mitochondrial targeting compound comprising a membrane-active fragment of gramicidin S or an E-alkylene isostere thereof conjugated to a cargo and references or describes additional moieties capable of intracellular delivery, including delivery to mitochondria.

US patent application 20160138015 describes oligonucleotides complementary to a non-coding chimeric mitochondrial RNA as well as compositions and kits comprising the same, and their use in treating and preventing metastasis or relapse of a cancer (e.g., a refractory HPV-associated cancer) in an individual previously treated for cancer with a therapy. Such embodiments may be used as an adjunct to or as a part of the embodiments enabled in accordance with the present invention.

Reevaluation and possible refinements to the therapies or moving to a subsequent round in planned therapy may be repeated as cells/mitochondria continue to develop/mutate. A modified therapy, preferably employing one or more changed target identification system, a changed gene targeted, a different membrane effect, etc. may be scheduled and be initiated according to a schedule based for example on time and/or might be based on an event, e.g., a schedule change, a weight gain or loss, a patient's or therapist's request, availability of a substance, etc.

Formulations suitable for transdermal administration can be presented as discrete patches adapted to remain in intimate contact with the epidermis of the recipient for a prolonged period of time. Formulations suitable for transdermal administration generally have active ingredients less than about 1.5 kd and may be delivered by iontophoresis (see, for example, Pharmaceutical Research 3(6):318 (1986)). These formulations typically take the form of an optionally buffered aqueous solution of the active compound. Suitable formulations comprise citrate or bis\tris buffer (pH 6) or ethanol/water and will normally contain from about 0.01 to 0.5M active ingredient, preferably about 0.05 to 0.33M, more preferably from about 0.1 to 0.2M. Depending on the active ingredient, the required dose, its solubility, etc., the concentration may vary for those in general use. Patches should not remain on the skin for lengthy time periods. In general a patch that provides a therapeutic dose over a period of from about 3 to about 7 days is desired. Daily patches can be used where desired. But beyond 7 days, there is risk that the skin under the patch may become irritated or diseased.

### EXAMPLES

### EARLY DETECTION, simply or complex machines

### Example A

An insect, such as a bee or fruit fly is observed to alter behavior or trained to respond to the proximity of one or more cancer cells in a differentiated pattern as compared to a non-cancer cell. The insect is secured for monitoring the firings of its individual nerves. Outputs for each nerve and/or a pattern of outputs from a nerve network are recorded and analyzed in proximity to at least one cancer cell. A second cancer source is brought in proximity to the insect nerves. A second pattern is noted. Similarities between patterns are noted for possible inclusion in a cancer indicative signature. Additional cancer sources are brought in proximity as desired until a satisfactory signature is produced.

A chemical analysis of the cancer emissions identifies individual VOCs or categories or classes of VOCs to be used in future tests. Output from exposure of the nervous array to one or more VOC is compared to the signature(s). Relevant VOCs are thereby identified.

An electronic array is designed and outfitted for sensitivity to the identified VOCs. The array may also contain sensor components whose positive detection output(s) will act as a subtractive element in a signature, even as an indicator of a totally negative result. Algorithms for individual cancers and various classes of cancers will report, presence, absence and/or probability assessments of the relevant cancer(s).

Treatments then proceed as desired.

### Example b

A mammal such as a rodent, canine or porcine vertebrate is trained to respond to a cancer. Multiple VOCs or mixtures of VOCs are tested for ability to elicit similar response. The VOC(s) are included in the cancer signature profile. The profile is refined using various mixtures at various ratios.

### Treatments

### Example 1

In this example a reovirus is modified to increase expression of bak/bax, when infecting a cell. The reovirus maintains its preferential ability to proliferate in ras activated cells thus sparing non-neoplastic cells. This example is preferably one step in a sequence of therapeutic steps that target different aspects of neoplastic activity.

### Example 2

This example uses a non-reovirus, e.g., a modified polio or herpes virus to target the cancer cells. A first embodiment is modified to increase expression of bak/bax. A second embodiment is modified to increase intracellular activity including, but not limited to: caspace activators, cytochrome c releasers, general permeability enhancers, ATPase inhibitors, K⁺ and/or Ca²⁺ permeabilizers, cytoskeleton disruptors, membrane disrupters, mitochondrial swelling agents, bax activators, bak activators, Bcl2 or BcIX reducers or inhibitors, lysosomal disrupters, oxidation damage, radical scavenger interference, reduced neutralization of active oxygens.

### Example 3

A prospective patient presents who has been pre-screened with a positive outcome for one or more nano markers relating to evidence of a precancerous or cancerous cell metabolism. A confirmatory screening confirms the initial pre-screen results, but also the confirmatory screen using a gas sensor array allows more precise identification and numeric scoring of the triggering nano markers. In this example, though not essential to benefit any individual patient, a chromatographic method, such as GC-MS chromatography, is used to search for possible additional nano markers. Collecting results from multiple patients is used to build an experiential database that can be used to: a) improve screening, for example adding additional channels to relevant gas sensor arrays; b) more precisely target metabolic changes in that individual patient; and c) grow the database that can become a basis guiding optimized treatment strategies for each individual presentation.

The chromatographic data for this patient suggest optimal first round targeting is supplementing mitochondria systemically with CoQ10 and attacking Bcl2 with a directed virus.

A second round is planned interfering with Drp1 expression to maintain mitochondrial networks.

### Example 4

A reovirus is modified to potentiate bak/bax while the organism is supplemented with at least one compound that potentiates apoptosis in general. Compounds such as tryptophan, omega 3, DCA, etc. when supplemented systemically will further tilt the balance of all cells exposed in favor of apoptosis. The cancer cells with stronger apoptotic influence will experience stronger effect.

### Example 5

At least two of the examples above or two modifications of one example are administered in sequence to an organism with a cancer. Preferably the sequence is scheduled, for example, the therapy is altered based on time, rather than observation of cells that have evolved to bypass the current therapy. The frequent change of the therapeutic compositions changes selective pressures acting on the evolving cancer cells. This undoes advantages gained through the survivability of the fittest evolutionary process in the tumor and by placing constantly changing pressures on the cells allows the organism's immune system better access to overcome the diminished capacities and load of the cancer cells.

### Example 6

A single or multiple vesicle liposome is prepared with a membrane sensitive to rupture at acidic pH. The contents, if unilamellar, or outer inter membrane, if multilamellar, aqueous phase includes a ligand that was chosen to preferentially bind a known cancer cell in the recipient. The ligand optionally a) enters the receptor bearing cell, b) lyses the membrane of the receptor bearing cell, or c) attaches to the receptor bearing cell. Under option a) the ligand may activate or inactivate one or more organelles or proteins that may be involved with cell death. Inactivation or swelling of mitochondria is one preferred embodiment. Under option b) the cell dies. Instances may occur where both a) and b) occur to varying proportions, leading to multiple outcomes leading to cell death. Under option c) attachment: may inactivate one or more cell receptors, transport proteins or pores; may inactivate or block one or more cell receptors, transport proteins or pores; may serve as a ligand to bind other substituents, for example a substituent on an inner lamella of the vesicle, contents of one or more lamellae of the vesicle, a hormone, an immune cell or molecule, a cytokine or other systemic molecule, a cell adhesion protein, a targeting micro- or nano-particle that may be chemically or physically activated, etc. These various options using vesicular bodies are examples of means the present invention may incorporate to effect cell death.

### Example 7

A cancer screen is performed on a patient. The cancer screen is designed to detect cells that either have progressed to a cancerous stage or have altered their metabolism to include reactions typical or leading to those of cancer cells.

The patient is then treated using one or several protocols featured in the present invention.

Since the treatment protocols of this invention are not as onerous as our standard cancer treatments, such as chemotherapeutic agents and/or radiation, it will often be appropriate to use the protocols of the present invention on patients who might otherwise appear healthy.

### Example 8

A person is diagnosed with cancer. A tumor biopsy is collected. The person, now patient, consults with one or more medical professionals and decides to treat the cancer, further deciding to try multiple therapeutic methods. The patient may consider that if one method is ineffective as many cancer therapies are, the next strategy may be effective. Each strategy knocking back the cancer buys more time. This patient also requests frequent assessments of therapeutic progress by analyzing biologic samples obtained every visit and sitting through a weekly MRI to monitor tumor growth or regression. These data may be useful for identifying which therapies prove efficacious and how efficacious each round might be.

Each round featured in this example may be considered as a separate example or a sub-example. While the featured invention will increase efficacy with multiple varying strategies over a period of time, ample benefit may be obtained employing any one round.

The first round commences with reovirus modified to transport both anti-Bcl2 genetic material, material that will be transcribed to interfere with Bcl2 expression and with RNA to be inserted into the host cell genome using reverse transcriptase machinery associated with retroviral infection. This patient receives two infusions the first week and two more at weekly intervals. Meanwhile the patient consumes a diet supplemented with l-tryptophan and omega 3 fats. At week 3 round two commences without waiting for patients samples to be interpreted. Round two uses a modified polio virus (modified to be non-infectious, to carry a surface protein that allows recognition and infection of the cancer cell, preferably a recognition site differing from that of the reovirus). This virus includes modifications that remove caspase inhibition and increase ceramide activity.

Round two therapy is repeated twice at weekly intervals when round three commences. Round three uses a modified herpes virus that specifically recognizes mutations fond in the cancer cells' mitochondrial genomes. Several weeks ago several mitochondrial DNA mutations had been noted and engineering of this herpes virus began. This virus targets mutated mtDNA specifically and a) shuts down duplication of the mitochondrial genome thereby locking in the present cohort of mtDNA rings. This virus also is engineered to interfere with at least one mitochondrial ATPase. The patient has meantime consumed a diet high in l-carnitine, α-lipoic acid and a trace of selenium.

Round four uses a modified erythrocyte membrane obtained from the patient. The membranes are flipped inside out and a tumor antigen receptor and membrane fusion proteins are incorporated into the membrane. This emptied cell is refilled with glycolysis inhibitors to shift the cancer cell metabolism towards oxidative phosphorylation and a decoupler to produce extra heat energy. The added heat encourages apoptosis as will the limited ATP. The limited ATP will further shift the cell towards apoptosis as the Ca⁺⁺ ATPase becomes less active, the mitochondria swell and cytochrome c is released into the cytoplasm.

The medical team has by now started serious analysis of the patient's MRI, metabolic and biologic sample data to assess which therapies are proving most fruitful against this cancer. For several more rounds therapies continue to change constantly changing evolutionary stresses upon the cancer. The biologic data obtained from the samples and images provided by the patient guide the team in determining how future rounds should be designed to optimize patient quality of life and therapeutic outcome.

Other examples or continuing rounds will include a means of targeting the therapy for improved effectiveness at the site of cancer cells. When a virus is used, the virus itself may lend an ability to kill a proportion of the cancer cells. Cargo, e.g., nucleic acid or protein that the virus is engineered to transport will preferably also be designed to interfere with cancer cell growth and/or proliferation and/or survival. Systemic supplements are optionally included in the patient's diet, as oral medication and/or co-infused with the virus or other carrier targeting the cancer. These strategies are continuously altered to provide ever changing stresses on the cancer cell thereby interfering with the cancer's evolutionary process enabling resistance to the anti-cancer compositions.

While these examples have included specifically named substances (reovirus, l-tryptophan, etc.), species of general group (Bcl2 of Bcl family, etc.), a numeric expression: such as a numeral (1,2, 3, etc.) or an order (first, second, etc.), these are for illustrative purposes and are not intended and should never be understood as features limiting the present invention.

One goal of this invention was to avoid or to reduce or to minimize toxic effect on non-neoplastic cells. But if the patient and medical team wish to incorporate one or more conventional interventions, one or more of these toxic agents, which some refer to as chemotherapy, can be incorporated into a round of the continuously changing anti-cancer therapies.

The present invention features embodiments including but not limited to:
In a cancer cell having one or a plurality of mutations in the mitochondrial DNA sequence of said cancer cell, delivering to said cancer cell a composition comprising at least one substance targeting at least one of the one or the plurality of mutations with the effect of causing death, destruction and/or disintegration of said cancer cell.
A neoplastic cell targeting virus, said virus having improved anti-neoplastic activity in part due to the virus comprising genetic material that facilitates apoptosis in a neoplastic cell.
An improved neoplastic cell targeting virus coding RNA material complementary to mtDNA that incorporates at least one mutation associated with the line of neoplastic cells being targeted.
An improved neoplastic cell targeting virus comprising genetic material inhibiting anti-apoptotic proteins.
An improved neoplastic cell targeting virus comprising genetic material that increases expression of one or more proteins selected from the group consisting of: Bcl2, BclXI, Mcl1, CED9, A1 and Bfl1.
An improved neoplastic cell targeting virus comprising genetic material comprising a BH domain, preferably a BH domain selected from the group consisting of: BH1 and BH2.
An improved neoplastic cell targeting virus with coding that induces expression of a fusion supporting protein, preferably a protein selected from the group consisting of: Mfn1, Mfn2 and Opa1.
An improved neoplastic cell targeting virus with coding that induces expression of an inhibiter of a fission supporting protein, preferably a protein selected from the group consisting of: Drp1, Mff, Fis1, GTPase, GTP synthase and MIEF1.
An improved neoplastic cell targeting virus with coding supporting activation of a pro-apoptotic protein or pathway. A protein may include, but not be limited to: a calpain, a caspase, a protein comprising BH3 domain, mitochondrial apoptosis-inducing protein (MAC), apoptosis inducing factor (AIF), protein kinase R (PKR), Bax, Bad, Bid and Bak. One effect of the protein activity may comprise release to or activation of cytochrome c in the cytosol.
An improved neoplastic cell targeting virus that facilitates mitochondrial swelling, possibly through increasing permeability of a membrane selected from the group consisting of: the outer mitochondrial membrane and the inner mitochondrial membrane.
An improved neoplastic cell targeting virus that facilitates mitochondrial swelling may work through inhibiting an ATPase, activating MPTP, uncoupling the ETC/ATP association, and/or increasing Ca⁺⁺ in the cytoplasm.
A method of killing a cancer cell comprising: i. a first targeted delivery to cancer cell of a first substance leading to apoptosis or necrosis of the cancer cell, ii. then, after a predetermined time has elapsed delivering to the cancer cell or surviving cancer cells a second substance leading to apoptosis or necrosis, the second substance differing the first substance and/or the second targeting mechanism differing from the first targeting mechanism.
A method of killing a cancer cell comprising delivering to a cancer cell containing organism, a substance selected from the group consisting of: one or more viruses as mentioned above in conjunction with healthy metabolism supportive components selected from the group consisting of: resveratrol, CoA. CoQ10, omega 3 fat, L-carnitine, α-lipoic acid, DCA, selenium, a flavone and a flavenoid. Embodiments of the virus may include one or more of those selected from the group consisting of: an engineered reovirus, an engineered vaccinia virus, an engineered herpes virus, an engineered vesicular stomatitis virus, an engineered senaca virus, an engineered Semliki Forest virus, an engineered ECHO and an engineered REGVIR virus, and an engineered polio virus.

1. A method for detecting presence of cancer comprising:
a. exposing an electronic sensor array to a biologic sample to obtain an output;
b. comparing said output from said array to a signature or signature library indicative of said at least one cancer; and
c. outputting a result based on said comparison, said result indicating presence of said at least one cancer or a probability of presence of said at least one cancer.

2. The method of claim 1 wherein said signature or signature library is derived from nervous responses of an invertebrate.

3. The method of claim 1 wherein said signature or signature library is derived from nervous responses of a vertebrate.

4. The method of claim 2 wherein said signature or signature library is derived from nervous responses of an insect.

5. The method of claim 4 wherein said signature or signature library is derived from nervous responses of an insect selected from the group consisting of a fruit fly and a bee.

6. The method of claim 3 wherein said signature or signature library is derived from nervous responses of a mammal.

7. The method of claim 6 wherein said mammal is selected from the group consisting of:
canines, porcines and rodents.

8. The method of claim 1 wherein at least on signature library is provided by:
a. situating a bioarray proximal to a plurality of biological sources to be assayed;
b. monitoring said bioarray to determine at least one response signature graded as positive for a proximity to a cancerous or precancerous cell in at least one member of said plurality and negative in the absence of proximal cancerous or precancerous cells;
c. exposing said bioarray to one or more VOC;
d. comparing response of said bioarray graded as positive to response of said bioarray to said one or more VOC;
e. adjusting VOC identity and/or ratio of the concentration of one of said one or more VOC to a second VOC of said one or more VOC to stimulate at least one signature response approximating at least one of said at least one response signature graded as positive;
f. providing an electronic sensor capable of reporting said adjusted VOC identity;
g. exposing a sample to said electronic sensor; and
h. obtaining output from said electronic sensor that indicates presence or fails to indicate presence of said adjusted VOC identity to insert in said library.

9. The method of claim 8 wherein said one or more VOC comprises one or more VOC selected from the group consisting of: aldehydes, ketones, alcohols, amino alcohols and polyamines.

10. The method of claim 8 further comprising repeating a through h to obtain a plurality of response signatures; compering said response signatures to one another; and compiling a library containing at least one signature composite indicative of a cancer generally.

11. The method of claim 8 wherein said signature composite is indicative of a cell metabolism predominated by glycolysis.

12. The method of claim 8 wherein said signature composite is indicative of a cell metabolism wherein the OXPHOS/glycolytic ration is decreased with respect to normal OXPHOS glycolytic ratios.

13. The method of claim 1 further comprising:
a. inhibiting the progression of cancer by:
b. subsequent to detection of at least one metabolic characteristic associated with a cell progressing towards cancer;
c. providing a therapeutic intervention to at least one cell in which said characteristic is present or suspected; said intervention selected from the group consisting of: a composition comprising at least one substance targeting at least one of the one or the plurality of mutations with the effect of causing death, destruction and/or disintegration of a cancer cell, neoplastic cell targeting virus, genetic material inhibiting anti-apoptotic proteins, genetic material supporting activation of a pro-apoptotic pathway, and a material facilitating mitochondrial swelling.

14. The method of claim 1 further comprising:
a. inhibiting the progression of cancer cells in a patient by:
b. using prescreen data indicative of a cancer nano marker to confirm suitability of said patient for further therapeutic intervention; and
c. providing therapeutic intervention to said patient, said intervention selected from the group consisting of: metabolism support compositions, metabolism altering compositions, mitochondrial fusion support, mitochondrial fission disruption, mitochondrial membrane disrupters, plasma membrane modifiers, and angiogenic inhibitors.

15. The method of claim 1 further comprising:
a. Improving cancer therapy by:
b. employing a nano marker database indicative of relations between said nano markers and one or more cancerous or precancerous condition,
c. comparing a nano marker profile presented by a patient with said database, and
d. designing a treatment strategy for said patient based on the contents of said database.

16. The method of claim 1 further comprising:
a. Improving cancer therapy by:
b. employing a nano marker database indicative of relations between said nano markers and one or more cancerous or precancerous condition,
c. comparing a nano marker profile presented by a patient with said database, and
d. implementing a treatment strategy for said patient based on the contents of said database.

17. A method of inhibiting proliferation of cancer cells, said method comprising:
a. identifying at least one mutation carried by a significant number of mitochondria in a population of cancer cells; making a compound specific to the mutated sequence; and delivering said compound to at least a portion of said population.
